SEQUENCE LISTING (10 sequences)

SEQ ID NO: 1 - DNA, 1050 bp, plasmodium falciparum
  CDS (1)..(1047)
atgaaacgctggtcaatcattacaggaatcgttataatattctgcatattaacatgtaaaggtcaagtagaaaacaaaaaggttgattttcgaacagaaaaaggaaagttcgtgcctttaaatctagtaccaggagatgttgtagaatattcgtgtccgtatagtttaaataatgatatccgaaatatgaatggggttgaacgagaacatttcgacaataaaaaattttgttttgattatatatttgttggaagtaagttaacatttttaaaagaatatgttcgtggttcttataatgtggtacataaagaagaagggaatttatatacatcacaatttagtgttcctcctgttgttttaacacatcgaaattttgattgtttttgttatatggaagaaaacaatgttgtggttaagaaggttttaagaatacatatatcaaatggtgttcttaggaaaataccgggatgtgattttaatgctgattataaagaatcgacagctataactacttttagtaatatgagtcctcgtcgtgtaaaagtatgtgacgtatatccgaagagtggagatttcataagtttaatgtgtccatcagattattcaattaaacctgatggttgtttttcaaatgtatatgtaaaaagatatcctaatgaagaagtgaaagaagaagatcgttttaatttgaatagaaaatgggatgcaagtaaatataatgttgtgtctatagaaacagttctaaaaatgaatatgataacacaaggtgataaatattctatattttcaaaattaccagatgtaaaagatcaagttgattttacatgtatttgtcaatcaaatgatgaacaagataatctaatgatgaatgtttatataaataacacatcttaccttacaaataatacaagaagtattggagttaacaagcattcattttctaattctgaaatttttgaaagaatagaaagagaagaaatttcatttgcgttttcttcctatttatcaataactttaattcttttatatcttttctttttaaacttttaa

SEQ ID NO: 2 - protein, 349 aa, plasmodium falciparum
MKRWSIITGIVIIFCILTCKGQVENKKVDFRTEKGKFVPLNLVPGDVVEYSCPYSLNNDIRNMNGVEREHFDNKKFCFDYIFVGSKLTFLKEYVRGSYNVVHKEEGNLYTSQFSVPPVVLTHRNFDCFCYMEENNVVVKKVLRIHISNGVLRKIPGCDFNADYKESTAITTFSNMSPRRVKVCDVYPKSGDFISLMCPSDYSIKPDGCFSNVYVKRYPNEEVKEEDRFNLNRKWDASKYNVVSIETVLKMNMITQGDKYSIFSKLPDVKDQVDFTCICQSNDEQDNLMMNVYINNTSYLTNNTRSIGVNKHSFSNSEIFERIEREEISFAFSSYLSITLILLYLFFLNF

SEQ ID NO: 3 - DNA, 1044 bp, plasmodium falciparum
  CDS (1)..(1041)
atgataaaattaagtaagaagtattgtttagggatatcctttgtattatatattttgttgtctgtttgtgaagggcataaaaatttaacatgtgactttaacgatgtatacaaattagaatttcatcctaatcaacaaacaagtgttactaaattatgtaatttaactcctaatgtattagaaaaggtaactataaaatgtggttcagataaattaaattataatttatatcctccaacttgttttgaagaggtatatgcatctaggaatatgatgcatttaaaaaaaataaaagagtttgtaatcggatcatcaatgtttatgagacgtagtttaacaccaaataaaattaacgaagtttctttcagaattccacctaatatgatgcctgaaaaacctatatattgtttttgtgaaaataaaaaaacaataactattaatggttccaatggaaatccttcaagtaaaaaagatataataaatagaggaatagttgaaattattataccttcattaaatgaaaaagttaaaggatgtgattttacaacaagcgaatctacaattttctcaaaaggatatagtattaatgaaatatctaataaatcatcaaataaccaacaagatattgtatgtacagttaaggcacatgctaatgatttaatcggatttaaatgtccaagcaattattctgttgaaccacatgattgttttgttagtgcattcaatttaagtgggaaaaatgaaaacctagaaaataaacttaaattaacaaatataattatggatcattataataatactttctattcaagattaccaagtttaatttctgataattggaaattcttttgtgtatgttcaaaagataatgaaaaaaaattagtctttaccgtagaagcaagcatttcatcaagtaatactaaacttgcttcaagagataatacataccaagattatatatccaactcttctttcttaaccctttcatcatattgtgccttcatcacttttattatcacatcattcttatcattcatcttataa

SEQ ID NO: 4 - protein, 347 aa, plasmodium falciparum
MIKLSKKYCLGISFVLYILLSVCEGHKNLTCDFNDVYKLEFHPNQQTSVTKLCNLTPNVLEKVTIKCGSDKLNYNLYPPTCFEEVYASRNMMHLKKIKEFVIGSSMFMRRSLTPNKINEVSFRIPPNMMPEKPIYCFCENKKTITINGSNGNPSSKKDIINRGIVEIIIPSLNEKVKGCDFTTSESTIFSKGYSINEISNKSSNNQQDIVCTVKAHANDLIGFKCPSNYSVEPHDCFVSAFNLSGKNENLENKLKLTNIIMDHYNNTFYSRLPSLISDNWKFFCVCSKDNEKKLVFTVEASISSSNTKLASRDNTYQDYISNSSFLTLSSYCAFITFIITSFLSFIL

SEQ ID NO: 5 - DNA, 1137 bp, plasmodium falciparum
  CDS (1)..(1134)
atgaagggtgttattttttgtttagttgttcttttatggagacaagcatgggtatcgagtaaaagtcacaaatgtgattttacaaaagagaaatatttattatctggagagaaggaagtatcttgtgaaatagatgcgaatccttcggatgatattacttttatatgtcctaataaaattgattcattatgttttcatactgtaaatatatctaaaaatataaatcaaaataagtcaacaatgtctatacaggatttattatatggttctgtagtatatggaaatactctttttatatctccttatgttagaacaaacacaccgttttattgtttttgtaatctggacacggtgacaatacaaaagtttttaaaaatcaataggttcctaaaagatgacgatgaattaagcgaagccgacgtgatgaaacacttaaaaggtgggaatgttagcgaggcacaagctgatgaatatttaaataaagctttaaatagatttaaaaaaatgaaggatctttcaaaattttttaatgatcaagcagataatacgaccaaattaaatctaccaaaatccttaaatattccaaatgatatattaaactatgatgtatataatagtagtaataatagaaatgatattgttgttaaagatgaagttacaaataagcaaattatttcaaaacgtggaataatgagtgtttttgttcgtagtaataataatgtaataaaaggttgtgattttggtaacaataataagaattatttttctcatcccatatctgtagcaggtaaggtaaataataaggtttgtaaaatacaaggaaaacctggagaattagttggatttaaatgtgcctttgaagaaaatggtaaggtcgaaccacctaattgttttgatcaagtattacataaaaataaagtaacggatttaaaaacattaattccaggatatgcttcttatacaaataaacacagttcaaaatatccatactatttaaaaataccacattttgttaacgaacaatatactattcaatgtaaatgtaaatcaaacaactcacagaatgaatacacttttgaattagatatacaacctggagaaagtgaagttgttcttaactcttttaagacgtcatga

SEQ ID NO: 6 - protein, 378 aa, plasmodium falciparum
MKGVIFCLVVLLWRQAWVSSKSHKCDFTKEKYLLSGEKEVSCEIDANPSDDITFICPNKIDSLCFHTVNISKNINQNKSTMSIQDLLYGSVVYGNTLFISPYVRTNTPFYCFCNLDTVTIQKFLKINRFLKDDDELSEADVMKHLKGGNVSEAQADEYLNKALNRFKKMKDLSKFFNDQADNTTKLNLPKSLNIPNDILNYDVYNSSNNRNDIVVKDEVTNKQIISKRGIMSVFVRSNNNVIKGCDFGNNNKNYFSHPISVAGKVNNKVCKIQGKPGELVGFKCAFEENGKVEPPNCFDQVLHKNKVTDLKTLIPGYASYTNKHSSKYPYYLKIPHFVNEQYTIQCKCKSNNSQNEYTFELDIQPGESEVVLNSFKTS

SEQ ID NO: 7 - DNA, 2391 bp, plasmodium falciparum
  CDS (1)..(2388)
atgtttgctgtaaacttaaaaatttgtatatttttgtcacttgtttcatttttgttacaatgtaagaatacacttgctaatgtaacttttgaacaaaaggtacagacaaatttaagtcatgataaaaatggagatatagtatatggacacagagaatttaaaggaggtatttatgcatttttaggatatgacaactgtacaatagaagttaataaaacagtgaatggaatagattggaatgaaaaaaaggacgtaaaagtaagtggtaataataatattgctgtagtatattccatttttacaagtgaagagaagatgatattaatttttaaatgtgataataaattttatattactaaatatggaaaagaatttaaatggtcagatcctaaagttatagatgtatctaatgttattggtacaaatactaccccagctgtttactctggttctttactatctatgaataatgattttgaaaaatatattttggtttgtgaaaatcatagtcagaattatataaatgtagtagaccaggaatatatgagagaaatacgtttattaggtaaatgtatgttatcttttgatgaaggaaataactggaagaatgaagtaatgaatttatatagtgatgaaggttatacaaaaattaatactttaagattatcagattacggaggaaaaatacttgtgaaaggaactaatgcacaaaatcttaaccaaacgatacgatccataattcttttatgtagtaatttgcatgattggaaattattttgtggacttcctaccataagattcaggaaggatatttcagtagaaaatctaacatatttaaatacttatcatctagccattgtaaaaaatgaagataaattacaattagcttttacatatgatttatttgaaacatttgatccgcaatatttgaacactgaatttaatggtgtttctcattattttgttttagctcctgatgaaatggtatatttattttatcatggaaatgagaaaaaaaattatgtcataaaaataaaaacagtaccaagaaaaataggttgtgaattaaatacaaatgatactgtaaataaaatttatacatacacgtataaatatatatataataataagctaagtgcgaaaacatgtaaagttccatcatcacatttaaaatattcgagtgatggtctatataaattatttgaggtaagattaccaaaagatataaaagtaactgaaaattgttttagatattctttcttgagtgatttaaataataaatatcatacaactatcataaaaacacgagttattaacaaattagaagattatgtagaagtacaatttcactttccgatttattatactaaatttttatacaattataaaagtacgtattgtgtcttgagtaataactatagaatagttgttgaatttgattatatacgtaatcatattgatctagattttccgtttgatacagatactgtaaagttatatagtaatgaaagtgttacacatgcatttagaaataatacagaaaaaacacatgtgcataaatttcctaaaggtacatatatgacatcttattttagttatgaaaaagaatatgttatatctaattatatagaagaaccattttctactacctttactatattaacccaaacacaaatgaatgttcattttatggctggtggacaaaaatataaatatgaaggtatcgatttaacagattcttcaccaaactatgaattatcacttaattctttgtcagatagccaaaatgttgatatttttgtatccaaatttgataataataaaacgataggttttgtttgtcctgttaaaagtagttatgacggattaaattgttttgacaacgtttatataaaaaataagacacttgtcaaaatcgaatatttatttggagaaaatgatatctttgttgttcctcaaaggagaatatataaaactgaaggtacagctatggaaagtcttttatatttaaataataacaatgtaaaaaaattaatagatgataaaagtataatacatttctattgtgaatgtaatgttaataataatgtaatcaaagttaattattatatatctccattttatgatgaaaatagtataaaacaagaaatcaacaaaaaagaccaagaaataactatgattaataaaactataccacaagatgaaaaagatattctattcaataatgaaaaagttgttcctttatcaaacgaacctcaagaaattattcaaccacctattcaggaaaaattaaatactactgatccttcaaaagcatatatatatggagctaatattatatttattgcaattatatctataatatccttgtctatttcttctttcatataa

SEQ ID NO: 8 - protein, 796 aa, plasmodium falciparum
MFAVNLKICIFLSLVSFLLQCKNTLANVTFEQKVQTNLSHDKNGDIVYGHREFKGGIYAFLGYDNCTIEVNKTVNGIDWNEKKDVKVSGNNNIAVVYSIFTSEEKMILIFKCDNKFYITKYGKEFKWSDPKVIDVSNVIGTNTTPAVYSGSLLSMNNDFEKYILVCENHSQNYINVVDQEYMREIRLLGKCMLSFDEGNNWKNEVMNLYSDEGYTKINTLRLSDYGGKILVKGTNAQNLNQTIRSIILLCSNLHDWKLFCGLPTIRFRKDISVENLTYLNTYHLAIVKNEDKLQLAFTYDLFETFDPQYLNTEFNGVSHYFVLAPDEMVYLFYHGNEKKNYVIKIKTVPRKIGCELNTNDTVNKIYTYTYKYIYNNKLSAKTCKVPSSHLKYSSDGLYKLFEVRLPKDIKVTENCFRYSFLSDLNNKYHTTIIKTRVINKLEDYVEVQFHFPIYYTKFLYNYKSTYCVLSNNYRIVVEFDYIRNHIDLDFPFDTDTVKLYSNESVTHAFRNNTEKTHVHKFPKGTYMTSYFSYEKEYVISNYIEEPFSTTFTILTQTQMNVHFMAGGQKYKYEGIDLTDSSPNYELSLNSLSDSQNVDIFVSKFDNNKTIGFVCPVKSSYDGLNCFDNVYIKNKTLVKIEYLFGENDIFVVPQRRIYKTEGTAMESLLYLNNNNVKKLIDDKSIIHFYCECNVNNNVIKVNYYISPFYDENSIKQEINKKDQEITMINKTIPQDEKDILFNNEKVVPLSNEPQEIIQPPIQEKLNTTDPSKAYIYGANIIFIAIISIISLSISSFI

SEQ ID NO: 9 - DNA, 2910 bp, plasmodium falciparum
  CDS (1)..(2907)
atgaaaataccgttttttattttacatattttattattacaatttttattatgtttaatacgttgttatgtgcacaatgatgtaataaaatttggagaagaaaattcgttaaaatgttcacaaggaaacctgtatgtgttacattgtgaagttcaatgtttgaatgggaataacgagataatacataaaagatgtaatgacgacatagaaaagaaatgtaatggtaataataaatgcatatatttttttgaatatgaacttcgaaaaaaaacacaatcctttcgaaataagaatagtatagaaataagtgaatgtgtagaatcggaacagaacgaagtaaaaacatctacgacttgtttattaagtaatagttttattttagatgaagcatttattcaatatttcttttttattaagaataagaatgaggaacctgttatttgtaaagatggtaatataaatattaaaagtgcacttttacattctcccttttgtgaaataaaattaaaagatatatcagaatatattagaaaaaaatgtgataataataaagaatgtttaatagatccattagatgtacagaaaaatttattaaatgaagaagatccatgttatattaataattcgtatgtgtctgttaatgttgtatgtaataaagaagaagaaattggagatgaaagtacagattctagttcaatggaaattcaggattctacatctaatgaacaagatgaaaatgttaaaggaatgagttcatcacaagaaatgaattctaataacgacgaaaataaaaatcaagataatgaaagtgatgatgatgttaataataataataataataataatgatgatcaagatgaacaaggtaatgatggtgatgttacctcttctatgaataaaaatgaagataacaaagatttagaacatggctcttcaaatgatgttaataataatacagacactcttgttaataataaagaaaataaagaatttgtattaaaagaaaaatcaagtttaacatctaaaattaataaagaattagcacatagaaccgctctttttaataaattagctgataatatatccttattattgaataaaaaatatgattccttcgaaattaaagatgtattagaagatagatataatgaaatgaaacgtgatgcaaatccagatgtatattacatttatttaatggatacattagatatagaaaaaatagaagatataaatttagaagaagttaaaatgtcattattagcttccttaaaagaaacaatgaataaaatagatactatagaaaagaaaattgaagaattcaaaaataaatatatttcattatataataaagtaaaaactaccatgccagaacttttcgatttaaatgaagatttagtattattatataatgatttccctttcgataacggtatgatatcatctgatatattctttaaatataatccaagtgaaaatattatggatcatcaggaaatggtaaaaaagggttctataacggaagacgaattaagaattgttaatgaccttgaaccattagataattatagaagaagaaaaagaattacagaattaagaaaaattcttgttgaaaaattaagaatcttatatttagaaaagaataacttatttaatacacaagctagttgtatcaaatcgtattgttacaagaacccattgaatctaaaaacactagaagtattattaaagaaaaattattatagattaaaagaaaataaagattatgatgttgtatcaagtattatacaacatcttgataatgtcgatgcaaacaagaaaaaaaaatggcttacacatgaaagaatacttaaaaaattacaagtacttatagctgaaggatataaaagaataaacgaaaaagaaaaagatatcgatagaagaatggctgtatataatgccctatatgaaaaagcacaatcttataatttacaaaaattatttaatgatagtaatgatttcttaaagaaatatgcaataatgggtaattcttttgatgatggagatgaagtattcggaagccaatcatccaactttaatatttttgatagtaataacactgatcaaaataatgaacaagaacaaccaaaacaagacgatcaattactaaataataataatgatgatgtcctatcagaatccaataacgaaaataaagagaaaacaagcgacgatgcaactcacaaagagacacaagaaaaatcagatcaagaaccttcacaaaatattcaagaagataatagtgatgaaaaacatgcagaaaatgaggaaaatgtagaacaaattgaaacagacagtaacgtttctgaagaagctaatgacgaaaataaagacaatatgcaaacaacaactgatgaaggaactgaagaattacaacaaaatgatgaagatgctgaatctctaacaaaggaaaattcaaaatcggaagaacaagaaaatgaagattcaacagatgctgaagctattgacaaagaagaagtagaaacagaagaaaaaggaaaagatgaacaaaaaaaagacgaacaaaaagaacaagatgaagaagaagatggcgaaaaagaaaataaacataaatcaagtgaaacaaccaacgaaacagtaactgacattgaagaaaataaaaatgaagtaaaaggtgaagaacacctacaaggatcagaacaatcaatagaagcatctgaatcatctcaaaaagatgaaactaaagaaacagaagataaagaagaatatgtaaatgcaaatgatgatgaatctagtgaagaagatacgactccaaatgaaacaaacaaaactgataatggcagttcatttttctttgctatgagtaatgcactcttagtaattttacttttattatttatagaattcctataa

SEQ ID NO: 10 - protein, 969 aa, plasmodium falciparum
MKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

(19) United States
(12) Patent Application Publication
(10) Pub. No.: US 2008/0107656 A1
(43) Pub. Date: May 8, 2008

(54) IMMUNOGENIC COMPOSITIONS AND METHODS OF USE THEREOF

(75) Inventors: Brendan Scott Crabb, Brunswick (AU); Paul Robert Sanders, Moonee Ponds (AU)

Correspondence Address: SEED INTELLECTUAL PROPERTY LAW GROUP PLLC, 701 FIFTH AVE, SUITE 5400, SEATTLE, WA, 98104, US

(73) Assignee: The Walter and Eliza Hall Institute of Medical Research, Parkville (AU)

(21) Appl. No.: 11/756,487

(22) Filed: May 31, 2007

Related U.S. Application Data
(60) Provisional application No. 60/810,054, filed on May 31, 2006.

(30) Foreign Application Priority Data
May 31, 2006 (AU) 2006202321

(51) Int. Cl. A61K 39/395; A61K 31/7088; A61P 33/02; C07H 21/00; C07K 16/00; C12N 15/63; C40B 40/08; C12N 1/00; C07K 14/00; A61P 33/06; A61K 38/16

(52) U.S. Cl. 424/141.1; 435/243; 435/320.1; 506/17; 514/12; 514/44; 530/350; 530/388.1; 530/389.1; 536/23.1

(57) ABSTRACT

The present invention relates generally to a method of eliciting or otherwise inducing an immune response to a Plasmodium microorganism and compositions for use therein. More particularly, the present invention relates to a method of eliciting or otherwise inducing an immune response to a Plasmodium microorganism by administering an immunogenic composition comprising one or more of Pf38, Pf12, Pf41, Pf92 or Pf113 or immunogenic fragment or homologue thereof. The present invention is useful, inter alia, as a prophylactic and/or therapeutic treatment for Plasmodium infections of mammals such as, for example, Plasmodium falciparum infection.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 940120_{—}405_SEQUENCE_LISTING.txt. The text file is 65 KB, was created on May 31, 2007, and is being submitted electronically via EFS-Web, concurrent with the filing of the specification.

### BACKGROUND

1. Technical Field

The present invention relates generally to a method of eliciting or otherwise inducing an immune response to a Plasmodium microorganism and compositions for use therein. More particularly, the present invention relates to a method of eliciting or otherwise inducing an immune response to a Plasmodium microorganism by administering an immunogenic composition comprising one or more of Pf38, Pf12, Pf41, Pf92 or Pf113 or immunogenic fragment or homologue thereof. The present invention is useful, inter alia, as a prophylactic and/or therapeutic treatment for Plasmodium infections of mammals such as, for example, Plasmodium falciparum infection.

2. Description of the Related Art

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Infection by the protozoan parasite Plasmodium falciparum results in several hundred million clinical cases of malaria each year of which approximately two million are fatal. The malaria parasite is considered to be one of the most serious infectious agents in the world, infecting 5% of the global population and causing serious mortality and morbidity to sensitive populations and hampering socio-economic development. Developing a vaccine to control human malaria is therefore a global health priority of enormous significance.

Progress toward this goal requires an understanding of the mechanisms that underpin both naturally acquired and vaccine-induced immunity. Antibodies that inhibit the growth of blood stage Plasmodium falciparum parasites in vitro are found in the sera of some, but not all, individuals living in malaria endemic regions [Marsh et al., 1989, Trans. R. Soc. Trop. Med. Hyg., 83:293-303; Brown et al., 1982, Nature, 297:591-593; Brown et al., 1983, Infect. Immun. 39:1228-1235; Bouharoun-Tayoun et al., 1990, J. Exp. Med. 172:1633-1641]. Inhibitory antibodies are likely to contribute to the clinical immunity observed in highly exposed individuals but their overall significance to protection remains unclear [Mohan et al., 1998, In Malaria, Parasite Biology, Pathogenesis and Protection, I. W. Sherman, Editor. ASM Press, Washington D.C. 467-493; McGregor et al., 1988, In Malaria, Principles and Practices of Malariology, W. H. Wernsdorfer and I. A. McGregor, Editors. Churchill Livingston, Inc., New York, 559-619]. Inhibitory antibodies generally function by preventing invasion of red blood cells by the extracellular merozoite form of the parasite.

Despite the significant body of knowledge which has been developed in relation to malarial infectivity and immune responsiveness, there is still not available a highly effective vaccine. Accordingly, there is an ongoing need to pursue the development of anti-malarial vaccine technology.

Most membrane proteins that coat the surface of the erythrocyte invasive merozoite form of the parasite are attached to the plasma membrane via a C-terminal glycosylphosphatidyl inositol (GPI) anchor. Many other proteins that are not directly membrane-associated are exported from the parasite and are potentially linked to the surface via interactions with GPI-anchored proteins. To date, 4 GPI-anchored merozoite surface proteins have been identified (MSP-1, -2, -4, -5) and 2 others (MSP-10 and RAMA) are present in organelles at the apical end of the parasite [Cowman et al., 2002, Science 298:126-128; Black et al., 2003, Mol. Biochem. Parasitol 127:59-68; Topolska et al., 2004, J. Biol. Chem. 279:4648-4656]. Another protein originally designated a merozoite surface protein, MSP-8, appears to instead reside in the ring-stage [Drew et al., 2005, Infect Immun. 73:3912-3922]. In contrast to the apical and peripheral classes of blood-stage antigen, the GPI-anchored proteins appear to be essential to blood-stage growth with repeated attempts to disrupt the genes encoding the 6 GPI-anchored proteins resulting in only one ‘knockout’, that of the MSP-5 gene [Cowman et al., 2005, in Molecular Approaches to Malaria, in press; Sanders et al., (2005) J. Biol Chem 280(48): 40169-40176]. This, together with their propensity to be targeted by host antibodies, places the merozoite GPI-anchored proteins amongst the most highly validated blood-stage vaccine targets. Nevertheless, these molecules have not proven to be highly effective vaccine candidates.

In work leading up to the present invention, there have been identified five novel protein molecules which exhibit unique potential as vaccines due to the fact that multiple points in the life cycle of Plasmodium can be targeted by the immune response which is thereby generated. One of the limitations inherent in the vaccine candidates proposed to date has been the fact that the antigens utilized to date have only been expressed at very specific stages of the malarial parasite life cycle and therefore have only facilitated the development of candidate vaccines with effect at quite narrow stages of the life cycle. This necessarily results in certain inherent immunological limitations in terms of the effectiveness of such a vaccine which is directed to an organism with a life cycle as complex as that of Plasmodium. Prior to the advent of the present invention, there had not been identified molecules which are expressed across a range of life cycle stages and it is only in light of this finding that it has been still further determined that the use of such molecules in fact facilitates a level of immune responsiveness which has not been obtainable to date.

### BRIEF SUMMARY

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains amino acid and nucleotide sequence information prepared using the program PatentIn Version 3.1, presented herein after the bibliography. Each amino acid and nucleotide sequence is identified in the sequence listing by the numeric indicator <210> followed by the sequence identifier (e.g., <210>1, <210>2, etc). The length, type of sequence (amino acid, DNA, etc.) and source organism for each sequence is indicated by information provided in the numeric indicator fields <211>, <212> and <213>, respectively. Amino acid and nucleotide sequences referred to in the specification are identified by the indicator SEQ ID NO: followed by the sequence identifier (e.g., SEQ ID NO:1, SEQ ID NO:2, etc.). The sequence identifier referred to in the specification correlates to the information provided in numeric indicator field <400> in the sequence listing, which is followed by the sequence identifier (e.g., <400>1, <400>2, etc). That is SEQ ID NO:1 as detailed in the specification correlates to the sequence indicated as <400>1 in the sequence listing.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

One aspect of the present invention provides a method of eliciting or inducing, in a mammal, an immune response directed to a Plasmodium microorganism said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins.

Another aspect of the present invention provides a method of eliciting or inducing, in a mammal, an immune response directed to a Plasmodium falciparum microorganism said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins.

Still another aspect of the present invention provides a method of eliciting or inducing, in a mammal, an immune response directed to a Plasmodium microorganism said method comprising administering to said mammal an effective amount of a composition which composition comprises one or more protein molecules selected from:

(i) Pf38; and optionally one or more of

(ii) Pf12;

(iii) Pf41;

(iv) Pf92; or

(v) Pf113;

for a time and under conditions sufficient to elicit or induce an immune response to said Pf38 and one or more of Pf12, Pf41, Pf92 or Pf113.

Yet still another aspect of the present invention provides a method of eliciting or inducing, in a mammal, an immune response directed to a Plasmodium microorganism said method comprising administering to said mammal an effective amount of a composition which composition comprises one or more protein molecules selected from:

(i) Pf12; and optionally one or more of

(ii) Pf38;

(iii) Pf41;

(iv) Pf92; or

(v) Pf13;

for a time and under conditions sufficient to elicit or induce an immune response to said Pf12 and one or more of Pf38, Pf41, Pf92 or Pf113.

Still yet another aspect of the present invention contemplates a method of therapeutically or prophylactically treating a mammal for a Plasmodium microorganism infection said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with the infection of said mammal by said Plasmodium.

A further aspect of the present invention contemplates a method of therapeutically or prophylactically treating a human for a Plasmodium falciparum infection said method comprising administering to said human an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with the infection of said human by said Plasmodium falciparum.

In another further aspect the present invention contemplates a method of therapeutically or prophylactically treating a mammal for a Plasmodium falciparum infection said method comprising administering to said mammal an effective amount of a composition which composition comprises Pf38 and, optionally, one or more of Pf12, Pf41, Pf92 or Pf113 for a time and under conditions sufficient to elicit or induce an immune response to said Pf38 and one or more of Pf12, Pf41, Pf92 or Pf113 wherein said immune response reduces, inhibits or otherwise alleviates any one or more symptoms associated with the infection of said mammal by said Plasmodium falciparum.

In yet another further aspect the present invention contemplates a method of therapeutically or prophylactically treating a mammal for a Plasmodium falciparum infection said method comprising administering to said mammal an effective amount of a composition which composition comprises Pf12, and, optionally, one or more of Pf38, Pf41, Pf92 or Pf113 for a time and under conditions sufficient to elicit or induce an immune response to said Pf12 and one or more of Pf38, Pf41, Pf92 or Pf113 wherein said immune response reduces, inhibits or otherwise alleviates any one or more symptoms associated with the infection of said mammal by said Plasmodium falciparum.

In a related aspect, the present invention provides a method for the treatment and/or prophylaxis of a mammalian disease condition characterized by a Plasmodium microorganism infection said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with said microorganism infection.

The present invention also contemplates a method for the treatment and/or prophylaxis of a human disease condition characterized by a Plasmodium falciparum infection said method comprising administering to said human an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with said Plasmodium falciparum infection.

Another aspect of the present invention relates to the use of a composition comprising one or more protein molecules selected from Pf38, Pf12, Pf41, Pf92 or Pf113 in the manufacture of a medicament for the therapeutic and/or prophylactic treatment of a mammalian disease condition characterized by a Plasmodium microorganism infection.

Yet another aspect of the present invention contemplates the use of a composition comprising one or more protein molecules selected from Pf38, Pf12, Pf41, Pf92 or Pf113 in the manufacture of a medicament for the therapeutic and/or prophylactic treatment of a human disease condition characterized by a Plasmodium falciparum infection.

In a related aspect the present invention is directed to a composition capable of inducing an immune response to Plasmodium, said composition comprising one or more protein molecules selected from Pf38, Pf12, Pf41, Pf92 or Pf113.

In yet another further aspect, the present invention contemplates a pharmaceutical composition comprising the composition as hereinbefore defined together with one or more pharmaceutically acceptable carriers and/or diluents.

Another aspect of the present invention is directed to antibodies to one or more of Pf38, Pf12, Pf41, Pf92 or Pf113 as hereinbefore defined.

Yet another aspect of the present invention relates to a pharmaceutical composition comprising an antibody directed to a protein of the present invention together with one or more pharmaceutically acceptable carriers or diluents as hereinbefore described.

A further aspect of the present invention relates to the use of the antibodies of the present invention in relation to disease conditions. For example, the present invention is particularly useful but in no way limited to use in treating Plasmodium infections, their symptoms and pathologies.

Another aspect of the present invention relates to a method of inhibiting, halting or delaying the onset of progression of a mammalian disease condition characterized by a Plasmodium infection said method comprising administering to said mammal an effective amount of an antibody as hereinbefore described.

In yet another aspect the present invention relates to the use of an antibody in the manufacture of a medicament for inhibiting, halting or delaying the onset or progression of a disease condition characterized by the infection of a mammal by a Plasmodium.

In a related aspect the present invention is directed to a method of reducing or alleviating the symptoms associated with a Plasmodium infection said method comprising administering to said mammal an effective amount of an antibody as hereinbefore described.

In yet another aspect the present invention relates to the use of an antibody in the manufacture of a medicament for reducing or alleviating the symptoms associated with the infection of a mammal by a Plasmodium.

Still another aspect of the present invention is directed to an isolated protein as set forth in SEQ ID NO:2, 4, 6, 8 or 10 or having at least about 80% or greater identity to SEQ ID NO:2, 4, 6, 8 or 10 across the length of the sequence or a functional or immunogenic fragment or homologue, thereof.

Still yet another aspect of the present invention is directed to a protein encoded by a nucleotide sequence as set forth in SEQ ID NOs:1, 3, 5, 7 or 9 or the sequence complementary to a sequence capable of hybridizing to SEQ ID NOs:1, 3, 5, 7 or 9 under low stringency conditions and which encodes an amino acid sequence as set forth in SEQ ID NOs:2, 4, 6, 8 or 10 or having at least about 80% or greater identity to SEQ ID NOs:2, 4, 6, 8 or 10 across the length of the sequence.

Yet still another aspect of the present invention is directed to an isolated nucleic acid selected from the list consisting of:

(i) An isolated nucleic acid molecule or functional fragment or homologue comprising a nucleotide sequence encoding, or complementary to a sequence encoding, an amino acid sequence substantially as set forth in SEQ ID NO:2, 4, 6, 8 or 10 or a functional or immunogenic fragment or homologue thereof, or an amino acid sequence having at least 80%, 90%, 95%, 96%, 97%, 98%, 99% or more sequence identity to SEQ ID NO:2, 4, 6, 8 or 10 over the length of the sequence, and/or a nucleic acid sequence capable of hybridizing to said nucleic acid molecule under low stringency conditions at 42° C.

(ii) An isolated nucleic acid molecule or functional or immunogenic fragment or homologue thereof comprising a nucleotide sequence encoding, or complementary to said sequence, wherein said nucleotide sequence is substantially as set forth in SEQ ID NO:1, 3, 5, 7 or 9 or a nucleotide sequence having at least about 80%, 90%, 95%, 96%, 97%, 98%, 99% or more identity over the length of the sequence of a nucleotide sequence capable of hybridizing to SEQ ID NO:1, 3, 5, 7 or 9 or complementary form thereof under low stringency conditions at 42° C.

(iii) An isolated nucleic acid molecule or derivative, homologue or analogue thereof comprising a nucleotide sequence as set forth in SEQ ID NO:1, 3, 5, 7 or 9.

### BRIEF DESCRIPTION OF THE SEVERAL VIEWS OF THE DRAWINGS

FIG. 1 is an image depicting GPI-anchored protein enrichment in DRM preparations. DRMs isolated from schizont-stage P. falciparum parasites (3D7 line) were floated on sucrose gradients. Panels show western blot analysis of individual fractions using antibodies specific for GPI-anchored, type 1 membrane and peripheral proteins as indicated. The bottom right panel represents the total protein as detected by silver staining and indicates the fractions that were combined to derive the four pools subjected to proteomic analysis. In most cases parasites were extracted with saponin prior to Triton X-100. However, saponin lysis was omitted in the preparation of parasite DRMs probed with SERA5 and SERA6 antibodies.

FIG. 2 is an image of the proteomic analysis of DRMs prepared from P. falciparum schizonts. The proteomes of pooled sucrose gradient DRM fractions prepared from whole (non-saponin) and saponin-treated (saponin) P. falciparum schizont-stage parasites were analyzed by MudPIT. The red boxes represent the peptide coverage in each pool for the identified proteins, the relative intensity of the color being proportional to the degree of peptide coverage (see Tables 1 and 2). The top 28 proteins on the saponin-treated list are expanded in the table (top right). The presence or absence of a predicted N-terminal signal sequence (SS) and/or the number of predicted transmembrane domain(s) (TM) are indicated. The asterisks indicate the presence of a predicted GPI-addition signal. Also shown is the rank position (as a percentile) by peptide coverage of each protein in the whole schizont proteome [Le Roch et al., 2004, Genome Res. 14:2308-2318] (Prot. %) and the timing of peak transcription (mRNA) in the life cycle [Le Roch et al., 2003, Science 301:1503-1508; Bozdech et al., (2003) PLoS Biol 1, E5]. LR, late rings; ET, early trophozoites; ES, early schizonts; LS, late schizonts; Sp, sporozoites; Gm, gametocytes.

FIG. 3 is an image of the validation of GPI-associated proteins. At left, identification of GPI-anchored proteins in P. falciparum schizont DRMs by labeling with ³H-glucosamine. Gel slices corresponding to several radiolabeled proteins (red) were excised and analyzed by LC-MS/MS. The percent peptide coverage detected for each protein is shown in brackets while assignments of other proteins is described in [Gilson et al., 2006, Mol. Cell Proteomics, April 7]. Right, immunoprecipitation of ³H-glucosamine-labeled schizont extracts with IgG isolated from P. falciparum-exposed individuals (Pool P). Reactive proteins are enriched in the bound fraction (+) whereas poorly reactive proteins were present in the flow through (−).

FIG. 4 is an image of the localization of Cys₆ proteins to the merozoite surface and apical organelles. (A) Surface localization of Pf38 and Pf12. Coding sequences were fused to a signal sequence GFP reporter gene in the inducible expression vector pTGPI-GFP as shown [Meissner et al., 2005, Proc. Natl. Acad. Sci. U.S.A. 102:2980-2985]. Once stably transformed populations were established, anhydrotetracycline was removed from the media to induce expression of the transgene. MS, mid-schizont; LS, late schizont; M, merozoite. (B) Surface localization of Pf41. Antisera raised to an MSP Pf41 fusion protein (Pf41 amino acids 115-229) recognize a protein of ˜40 kDa in P. falciparum (3D7) parasites that were either saponin-lysed (S) or untreated (NS) prior to solubilization in non-reducing sample buffer. The reactivity of mouse anti-Pf41 antibodies with schizonts and free merozoites in a double labeling immunofluorescence assay with rabbit anti-MSP1 antibodies is shown. The arrows indicate the apical end of the parasite (dense structures).

FIG. 5 is an image depicting the reaction of P. falciparum merozoite Cys₆ proteins with antibodies in human immune sera. (A) Parasites expressing MSP-2, MSP-7, Pf41, Pf38, and Pf12 GFP fusion proteins under the inducible promoter system [Meissner et al., 2005, Proc. Natl. Acad. Sci. U.S.A., 102: 2980-2985] were cultured to the schizont-stage and subjected to immunoprecipitation (IP) with IgG prepared from pooled sera from Papua New Guinean adults (Pool M [O'Donnell et al., 2001, J. Exp. Med., 193: 1403-1412]). IgG from pooled non-immune donors was used as a negative control. Following immunoprecipitation, samples were analyzed by Western probing with mouse anti-GFP antibodies. Pf38-GFP and Pf12-GFP were strongly and specifically associated with the Pool M IgG and were depleted in the unbound fraction (Flow through), whereas MSP-2 and MSP7-like (MSP7L) proteins did not appear to react strongly with this reagent. The Pf41-GFP fusion was included as a negative control. (B) Western blot of an MBP-Pf41 fusion protein (Pf41 amino acids 115-229) probed with human anti-P. falciparum IgG.

FIG. 6 is an image depicting the localization of Pf92 to the merozoite surface. The full-length gene encoding Pf92 was fused to GFP and expressed in the inducible expression vector pJGPI-GFP. Prior to microscopy, parasites were incubated in culture medium containing 100 ng/ml 4′,6-diamidino-2-phenylindole (DAPI). Pf92-GFP localized at the merozoite surface was also visualized using phase contrast microscopy (+phase).

FIG. 7 is a schematic representation depicting DRM surface and apical proteins identified in P. falciparum merozoites. All proteins are drawn to scale and those in red are newly identified in this study. Shaded regions constitute cysteine-rich regions and the diamonds and ovals represent individual 6-cys and EGF-like domains respectively. SERA5 and 6 are lost from DRMs following saponin lysis. Indicated in the merozoite are the apical rhoptry organelles (R), the inner membrane (IM) and the nucleus (N). The inset highlights some of the other DRM proteins potentially associated with the surface and/or apical organelles.

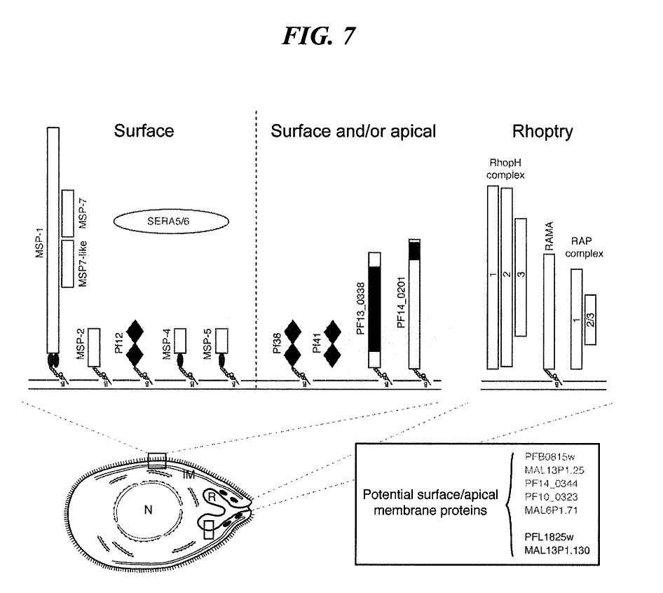

FIG. 8 is a schematic representation of the Plasmodium falciparum six-cysteine surface antigen family. The 10 known members of the P. falciparum 6-cys family are shown and each is color coded according to its transcription in blood-stages, gametocytes and/or sporozoites [Le Roch et al., 2003, supra]. Note that not all are GPI-anchored.

FIG. 9 is an image of the transcriptional profile for genes encoding the GPI-associated proteins. Microarray data for 12 of the 13 GPI-associated genes (NB: no data is available for RAMA) is shown for glass slide competitive array (left) [Bozdech et al., 2003, supra] and Affymetrix array (right) [Le Roch et al., 2003, supra]. For the glass slide array, yellow represents time-points in the blood-stage cycle that the gene of interest were increased in expression whereas blue represents the times at which they were decreased. This data shows that all genes except for PF14_{—}0201 are co-regulated and are most strongly expressed late in the cycle. With respect to the affymetrix array, the total expression intensity in blood, sporozoite and gametocytes is represented by the different colored bars. It was evident from this analysis that Pf38 is particularly strongly expressed in all 3 lifecycle stages.

### DETAILED DESCRIPTION

The present invention is predicated, in part, on the identification of a panel of surface protein molecules, of which several are expressed across a number of the different life cycle stages of the Plasmodium parasite. Still further, it has been determined that vaccines comprising these molecules are, in fact, immunologically more effective than vaccines which utilize the currently known Plasmodium derived antigens. This determination has therefore facilitated the development of compositions and methodology for application, inter alia, in the prophylactic or therapeutic treatment of Plasmodium infection.

Accordingly, one aspect of the present invention provides a method of eliciting or inducing, in a mammal, an immune response directed to a Plasmodium microorganism said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins.

The “mammal” as described herein includes humans, primates, livestock animals, (e.g., sheep, pigs, cows, horses, donkeys), laboratory test animals (e.g., mice, rats, rabbits, guinea pigs), companion animals (e.g., dogs, cats), captive wild animals (e.g., foxes, kangaroos, deer), aves (e.g., chicken, geese, ducks, emus, ostriches), reptiles or fish. Preferably, the mammal is a human.

“Malaria” is a term used to describe a class of diseases which are caused by infection with the protozoans of the genus Plasmodium. These diseases are also known by other names including Ague, Marsh Fever, Periodic Fever and Paludism. Accordingly, reference to “Plasmodium microorganism” should be understood as a reference to any Plasmodium species. Such as, but not limited to, the Plasmodium species P. falciparum, P. malariae, P. ovale and P. vivax, P. yoelii, P. berhei, P. chabaudi, P. knowlesi, P. reichnowi, P. simium, P. fieldi, P. simiovale, P. cyanomolgi, P. hylobati, P. inui, P. gonderi, P. gallinaceum, P. elongatum. In a particularly preferred embodiment, the Plasmodium species are P. falciparum, P. malariae, P. ovale and P. vivax which, without limiting the present invention to any one theory or mode of action, are known to infect humans. In general, and without limiting the present invention in any way, the disease is transmitted by the Anopheles mosquito and is confined mainly to tropical and sub-tropical areas. Parasites in the blood of an infected person are taken up into the stomach of the mosquito as it feeds. Here, they multiply and then invade the mosquito salivary glands. When the mosquito bites a subject, parasites are simultaneously injected into the blood stream and thereafter migrate to the liver and other organs, where they multiply. After an incubation period varying from 12 days (P. falciparum) to 10 months (some varieties of P. vivax), parasites return to the blood stream and invade the red blood cells. Rapid multiplication of the parasites results in destruction of the red cells and the release of more parasites capable of infecting other red cells. This causes a short bout of shivering, fever and sweating and the loss of healthy red cells results in anemia. When the next batch of parasites is released, symptoms reappear. The interval between fever attacks varies in different forms of malaria. For example, in Quartan malaria the interval is approximately three days and is caused by the species P. malaria. In Tertian malaria, the interval is two days and is caused by the species P. ovale and P. vivax. In malignant Tertian malaria, this being the most severe form of malaria, the interval is from a few hours to two days. This form of malaria is caused by P. falciparum.

Still without limiting the present invention in any way, the primitive malarial parasites which are injected by the mosquito are termed sporozoites. These sporozoites circulate in the blood for a short time and then settle in the liver where they enter the parenchymal cells and multiply. This stage is known as the pre-erythrocytic schizogony. After multiplication, there may be thousands of young parasites known as merozoites in one liver cell. At this time, the liver cell ruptures and the free merozoites enter red blood cells. In the red blood cells, the parasites develop into two forms, a sexual and an asexual cycle. The sexual cycle produces male and female gametocytes which circulate in the blood and are taken up by a female mosquito when taking a blood meal. In the asexual cycle, the developing parasites form schizonts in the red blood cells which contain many merozoites. The infected red cells rupture and release a batch of young merozoites which invade new red cells. The species P. vivax, P. ovale and P. malariae develop in the peripheral blood subsequently to the liver cycles. However, in the case of P. falciparum only ring forms and gametocytes are present in the peripheral blood.

Accordingly, it should be understood that Plasmodium parasites pass through a number of developmental stages through their life cycle. Reference to “Plasmodium microorganism” should therefore also be understood to include reference to the microorganism at any one of its life cycle developmental stages, whether that be a mature or immature developmental stage.

Preferably, said Plasmodium microorganism is a Plasmodium falciparum microorganism and said mammal is a human.

The present invention therefore preferably provides a method of eliciting or inducing, in a mammal, an immune response directed to a Plasmodium falciparum microorganism said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins.

Preferably, said mammal is a human.

As detailed hereinbefore, a novel panel of Plasmodium derived merozoite surface antigens have been identified which exhibit unique functionality in terms of their use in a vaccine due to the fact that they enable the targeting of multiple life stages of the Plasmodium parasite. In particular, Pf38, Pf12 and Pf41 are comprised of dual “six-cysteine” domains, a fold found in a family of Plasmodium proteins, some of which localize to the surface of gametocyte stage parasites. Pf92 and Pf113 represent two other novel GPI anchored proteins which have been identified in the context of the present invention. Without limiting the present invention to any one theory or mode of action, these proteins are recognized well by antibodies in naturally exposed individuals highlighting their utility as antigens. Moreover, both Pf12 and Pf38 display broad expression across the different life cycle stages, most notably Pf38, which is expressed strongly in schizont, gametocyte and sporozoites stages. Accordingly, there is now provided, for the first time, a recombinant form of a single protein which exhibits broad utility as a blood-stage, transmission blocking and pre-erythrocytic vaccine.

To this end, it should be understood that Pf38, Pf12, Pf41, Pf92 and Pf113 proteins are defined by SEQ ID NOs:2, 4, 6, 8 and 10, respectively, while Pf38, Pf12, Pf41, Pf92 and Pf113 cDNA molecules are defined by SEQ ID NOs:1, 3, 5, 7 and 9.

Preferably, the composition of the present invention comprises Pf38 and optionally one or more of Pf12, Pf41, Pf92 and/or Pf13.

In another preferred embodiment, the present invention comprises Pf12 and optionally one or more of Pf38, Pf41, Pf92 and/or Pf113.

There is therefore preferably provided a method of eliciting or inducing, in a mammal, an immune response directed to a Plasmodium microorganism said method comprising administering to said mammal an effective amount of a composition which composition comprises one or more protein molecules selected from:

(i) Pf38; and optionally one or more of

(ii) Pf12;

(iii) Pf41;

(iv) Pf92; or

(v) Pf13;

for a time and under conditions sufficient to elicit or induce an immune response to said Pf38 and one or more of Pf12, Pf41, Pf92 or Pf113.

In another preferred embodiment, there is provided a method of eliciting or inducing, in a mammal, an immune response directed to a Plasmodium microorganism said method comprising administering to said mammal an effective amount of a composition which composition comprises one or more protein molecules selected from:

(i) Pf12; and optionally one or more of

(ii) Pf38;

(iii) Pf41;

(iv) Pf92; or

(v) Pf113;

for a time and under conditions sufficient to elicit or induce an immune response to said Pf12 and one or more of Pf38, Pf41, Pf92 or Pf113.

Preferably, said composition comprises Pf38 alone, Pf12 alone or both of Pf38 and Pf12.

More preferably, said Plasmodium is Plasmodium falciparum and said mammal is a human.

Reference to “Pf38”, “Pf12”, “Pf41”, “Pf92” and “Pf113” should be understood as a reference to all forms of these molecules and to immunogenic fragments, derivatives, homologues and variants thereof. The panel of Pf molecules which have been identified by the inventors are defined in more detail hereinafter in this specification. However, it should be understood that in the context of the present aspect of the invention, one may either utilize any immunogenic form of these molecules including any isomeric forms which may arise from an alternative splicing of Pf38, Pf12, Pf41, Pf92 or Pf113 mRNA. It should also be understood to extend to these molecules even in dimeric, multimeric or fusion protein form.

These proteinaceous molecules may be derived from any suitable source such as natural, recombinant or synthetic sources.

“Derivatives” of the molecules herein described include immunogenic fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, immunogenic regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, these molecules may be fused to a molecule to facilitate its localization to a particular site, such as a lymph node. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

A “variant” should be understood to mean molecules which exhibit at least some of the immunogenicity of the form of the molecule of which it is a variant. A variation may take any form and may be naturally or non-naturally occurring.

The proteins of the invention, as defined above, include all “mimetic” and “peptidomimetic” forms. The terms “mimetic” and “peptidomimetic” refer to a synthetic chemical compound which has substantially the same structural and/or functional characteristics of the proteins of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with proteins of the invention which are conservative variants, routine experimentation will determine whether a mimetic is within the scope of the invention, i.e., that its structure and/or function is not substantially altered.

Protein mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A protein can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thienylalanine; D- or L-1, -2,3-, or 4-pyrenylalanine; D- or L-3 thienylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylalinines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimethylpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

The skilled artisan will recognize that individual synthetic residues and proteins incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

A component of a protein of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R— or S— form.

The invention also provides proteins that are “substantially identical” to the proteins of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine).

Reference to “elicit or induce an immune response” should be understood as a reference to stimulating or facilitating the stimulation of a specific immune response. This specific immune response may be a T cell and/or a humoral response which is directed to any one or more epitopes of the Pf molecules herein defined. It should be understood, however, that there may or may not also occur a non-specific immune response.

An “effective amount” means an amount necessary at least partly to attain the desired immune response, or to prevent or to delay the onset or inhibit progression or halt altogether, the onset or progression of a particular condition being treated. This amount varies depending upon the health and physical condition of the individual to be treated, the taxonomic group of the individual to be treated, the capacity of the individual's immune system to stimulate a specific immune response, the degree of protection desired, the formulation of the vaccine, the assessment of the medical situation, and other relevant factors. It is expected that the amount will fall in a relatively broad range that can be determined through routine trials.

A further aspect of the present invention relates to the use of the invention in relation to disease conditions. For example, the present invention is particularly useful, but in no way limited to use in therapeutically or prophylactically treating Plasmodium infections such as by immunizing a mammal against a Plasmodium infection. In this regard, it should be understood that the method of the present invention is directed to inducing an immune response for the purpose of alleviating or preventing the onset of symptoms associated with a Plasmodium infection (such as toxicity and immunosuppression) or reducing or preventing Plasmodium infection. Reference herein to “symptoms” associated with Plasmodium infection should be understood to extend to both the infection itself as well as the physical and/or physiological consequences (such as toxicity or immunosuppression) of such an infection.

Accordingly, another aspect of the present invention contemplates a method of therapeutically or prophylactically treating a mammal for a Plasmodium microorganism infection said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with the infection of said mammal by said Plasmodium.

The mammal undergoing treatment may be a human or animal in need of therapeutic or prophylactic treatment for a disease condition or a potential disease condition. Preferably, said mammal is a human.

More particularly, the present invention contemplates a method of therapeutically or prophylactically treating a human for a Plasmodium falciparum infection said method comprising administering to said human an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with the infection of said human by said Plasmodium falciparum.

Without limiting this aspect of the present invention, administration of said composition may act to result in the production of antibodies which either prevent manifestation of symptoms such as toxicity and immunosuppression or which affect the parasite directly, for example by killing the parasite (at any stage of its life cycle) via binding to its surface and inhibiting its growth, development or the onward progression of the overall infection.

In one preferred embodiment, the present invention contemplates a method of therapeutically or prophylactically treating a mammal for a Plasmodium falciparum infection said method comprising administering to said mammal an effective amount of a composition which composition comprises Pf38 and, optionally, one or more of Pf12, Pf41, Pf92 or Pf113 for a time and under conditions sufficient to elicit or induce an immune response to said Pf38 and one or more of Pf12, Pf41, Pf92 or Pf113 wherein said immune response reduces, inhibits or otherwise alleviates any one or more symptoms associated with the infection of said mammal by said Plasmodium falciparum.

In another preferred embodiment the present invention contemplates a method of therapeutically or prophylactically treating a mammal for a Plasmodium falciparum infection said method comprising administering to said mammal an effective amount of a composition which composition comprises Pf12, and, optionally, one or more of Pf38, Pf41, Pf92 or Pf113 for a time and under conditions sufficient to elicit or induce an immune response to said Pf12 and one or more of Pf38, Pf41, Pf92 or Pf113 wherein said immune response reduces, inhibits or otherwise alleviates any one or more symptoms associated with the infection of said mammal by said Plasmodium falciparum.

According to these preferred embodiments, said mammal is preferably a human.

Most preferably, said composition comprises Pf38 alone, Pf12 alone or both Pf38 and Pf12.

Reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of onset of a particular condition. “Treatment” may also reduce the severity of an existing condition or the frequency of acute attacks (for example, reducing the severity of initial infection).

In accordance with these methods, the composition defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In a related aspect, the present invention provides a method for the treatment and/or prophylaxis of a mammalian disease condition characterized by a Plasmodium microorganism infection said method comprising administering to said mammal an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with said microorganism infection.

More particularly, the present invention contemplates a method for the treatment and/or prophylaxis of a human disease condition characterized by a Plasmodium falciparum infection said method comprising administering to said human an effective amount of a composition which composition comprises one or more protein molecules selected from Pf38, Pf12, Pf41, Pf92 or Pf113 for a time and under conditions sufficient to elicit or induce an immune response to one or more of said proteins wherein said immune response reduces, inhibits or otherwise alleviates any one or more symptoms associated with said Plasmodium falciparum infection.

Preferably, said disease condition is malaria and said mammal is a human.

More preferably, said composition comprises:

(i) Pf38 and, optionally, Pf12, Pf41, Pf92 or Pf113;

(ii) Pf12 and, optionally, Pf38, Pf41, Pf92 or Pf113;

(iii) Pf38 and Pf12;

(iv) Pf38; or

(v) Pf12.

Administration of a composition of the present invention in the form of a pharmaceutical composition, may be performed by any convenient means. The proteins of the pharmaceutical composition are contemplated to exhibit therapeutic activity when administered in an amount which depends on the particular case. The variation depends, for example, on the human or animal and the proteins chosen. A broad range of doses may be applicable. Considering a patient, for example, from about 0.1 μg to about 1 mg of the composition may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The composition may be administered in a convenient manner such as by the oral, intravenous (where water soluble), respiratory, transdermal, intraperitoneal, intramuscular, subcutaneous, intradermal or suppository routes or implanting (e.g., using slow release molecules). The composition may be administered in the form of pharmaceutically acceptable nontoxic salts, such as acid addition salts or metal complexes, e.g., with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, transdermally, intratracheally, nasopharyngeally, intravenously, intraperitoneally, subcutaneously, intracranially, intradermally, intramuscularly, intraoccularly, intrathecally, intracerebrally, intranasally, infusion, orally, rectally, via IV drip, patch and implant.

Another aspect of the present invention relates to the use of a composition comprising one or more protein molecules selected from Pf38, Pf12, Pf41, Pf92 or Pf113 in the manufacture of a medicament for the therapeutic and/or prophylactic treatment of a mammalian disease condition characterized by a Plasmodium microorganism infection.

More particularly, the present invention contemplates the use of a composition comprising one or more protein molecules selected from Pf38, Pf12, Pf41, Pf92 or Pf113 in the manufacture of a medicament for the therapeutic and/or prophylactic treatment of a human disease condition characterized by a Plasmodium falciparum infection.

Preferably, said disease condition is malaria.

More preferably, said composition comprises:

(i) Pf38 and, optionally, Pf12, Pf41, Pf92 or Pf113;

(ii) Pf12 and, optionally, Pf38, Pf41, Pf92 or Pf113;

(iii) Pf38 and Pf12;

(iv) Pf38; or

(v) Pf12.

The present invention should also be understood to extend to the immunogenic compositions hereinbefore defined.

Accordingly, in a related aspect the present invention is directed to a composition capable of inducing an immune response to Plasmodium, said composition comprising one or more protein molecules selected from Pf38, Pf12, Pf41, Pf92 or Pf113.

Preferably, said composition comprises:

(i) Pf38 and, optionally, Pf12, Pf41, Pf92 or Pf113;

(ii) Pf12 and, optionally, Pf38, Pf41, Pf92 or Pf113;

(iii) Pf38 and Pf12;

(iv) Pf38; or

(v) Pf12.

In yet another further aspect, the present invention contemplates a pharmaceutical composition comprising the composition as hereinbefore defined together with one or more pharmaceutically acceptable carriers and/or diluents. Said agents are referred to as the active ingredients.

Still another aspect of the present invention provides the compositions as hereinbefore defined for use in therapy.

The modulatory agents of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of modulatory agents of the invention are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

The composition of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix (1996) Pharm Res. 13:1760-1764; Samanen (1996) J. Pharm. Pharmacol. 48:119-135; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani (1996) “Systemic delivery of peptides and proteins across absorptive mucosae” Crit. Rev. Ther. Drug Carrier Syst. 13:85-184. For topical, transdermal administration, the agents are formulated into ointments, creams, salves, powders and gels. Transdermal delivery systems can also include, e.g., patches.

The composition of the invention can also be administered in sustained delivery or sustained release mechanisms, which can deliver the formulation internally. For example, biodegradable microspheres or capsules or other biodegradable polymer configurations capable of sustained delivery of a peptide can be included in the formulations of the invention (see, e.g., Putney (1998) Nat. Biotechnol. 16:153-157).

For inhalation, the composition of the invention can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The composition of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun (1997) Anesth Analg. 85:317-323). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's,. See also, Bai (1997) J. Neuroimmunol. 80:65-75; Warren (1997) J. Neurol. Sci. 152:31-38; Tonegawa (1997) J. Exp. Med. 186:507-515.

In one aspect, the pharmaceutical formulations comprising compositions of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble modulatory agents of the invention have been attached to the surface of the monolayer or bilayer. For example, peptides can be attached to hydrazide-PEG-(distearoylphosphatidyl)ethanolamine-containing liposomes (see, e.g., Zalipsky (1995) Bioconjug. Chem. 6:705-708). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla (1996) J. Pharm. Sci. 85:5-8), transmucosally, or orally. The invention also provides pharmaceutical preparations in which the nucleic acid, peptides and/or polypeptides of the invention are incorporated within micelles and/or liposomes (see, e.g., Suntres (1994) J. Pharm. Pharmacol. 46:23-28; Woodle (1992) Pharm. Res. 9:260-265). Liposomes and liposomal formulations can be prepared according to standard methods and are also well known in the art, see, e.g., Remington's; Akimaru (1995) Cytokines Mol. Ther. 1:197-210; Alving (1995) Immunol. Rev. 145:5-31; Szoka (1980) Ann. Rev. Biophys. Bioeng. 9:467, U.S. Pat. Nos. 4,235,871, 4,501,728 and 4,837,028.

The pharmaceutical compositions of the invention can be administered in a variety of unit dosage forms depending upon the method of administration. Dosages for typical modulatory pharmaceutical compositions are well known to those of skill in the art. Such dosages are typically advisorial in nature and are adjusted depending on the particular therapeutic context, patient tolerance, etc. The amount of modulatory agent adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton (1997) “Bioavailability and transport of peptides and peptide drugs into the brain” Peptides 18:1431-1439; Langer (1990) Science 249:1527-1533.

The terms “compound”, “active agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

The pharmaceutical composition may also comprise genetic molecules such as a vector capable of transfecting target cells where the vector carries a nucleic acid molecule capable of expressing, for example, one or more of the protein molecules herein defined. The vector may, for example, be a viral vector and it may be administered by any suitable method including, for example transfection directly into the cells of the mammal being treated or transfection into a host cell, such as a bacterium, yeast or attenuated parasite, which is then introduced into the mammal. In a particularly preferred embodiment, and in the context of the treatment of humans, said protein molecules are expressed on the surface of an innocuous rodent parasite (such as P. berghei). This effectively renders the genetically modified P. berghei more P. falciparum—like at the antigenic level. Such a microorganism will exhibit excellent immunogenicity but without the side effects and infectivity which would be associated with the administration of P. falciparum to a human. Said genetically modified P. berghei could be administered either live or killed.

Administration of the protein molecules of the present invention induces antibody production and in particular IgG production. These antibodies are involved in inhibiting, halting or delaying the onset or progression of symptoms associated with Plasmodium infection such as, for example, pathological responses to Plasmodium infection. The antibodies function, for example, by direct antiparasitic effect such as killing the parasite by binding to its surface and inhibiting its growth or development or otherwise inhibiting its onward progression. Antibodies directed to these proteins may therefore also be utilized in treating Plasmodium infections therapeutically or prophylactically.

Accordingly, another aspect of the present invention is directed to antibodies to one or more of Pf38, Pf12, Pf41, Pf92 or Pf113 as hereinbefore defined.

Such antibodies may be monoclonal or polyclonal, may be of any isotype and may be selected from naturally occurring antibodies or may be specifically raised.

The antibodies of the present invention are particularly useful as therapeutic or prophylactic agents. Fragments of antibodies may be used such as Fab fragments. Furthermore, the present invention extends to recombinant and synthetic antibody, to an antibody hybrid and to humanized antibody. A “synthetic antibody” is considered herein to include fragments and hybrids of antibodies. The antibodies of this aspect of the present invention are particularly useful for immunotherapy and immunoprophylaxis and may also be used as a diagnostic tool for assessing, for example, parasitic infection or for monitoring the progress of therapeutic regimen.

It is within the scope of this invention to include any second antibodies (monoclonal, polyclonal or fragments of antibodies or synthetic antibodies) directed to the first mentioned antibodies discussed above. Both the first and second antibodies may be used in detection assays or a first antibody may be used with a commercially available anti-immunoglobulin antibody.

Both polyclonal and monoclonal antibodies are obtainable by immunization with the subject protein molecule. The methods of obtaining both types of sera are well known in the art and are described in more detail hereinafter. Polyclonal sera are less preferred but are relatively easily prepared by injection of a suitable laboratory animal with an effective amount of the subject protein, or antigenic parts thereof, collecting serum from the animal, and isolating specific sera by any of the known immunoabsorbent techniques. Although antibodies produced by this method are utilizable in virtually any type of application, they are generally less favored because of the potential heterogeneity of the product.

The use of monoclonal antibodies is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Yet another aspect of the present invention relates to a pharmaceutical composition comprising an antibody directed to a protein of the present invention together with one or more pharmaceutically acceptable carriers or diluents as hereinbefore described.

A further aspect of the present invention relates to the use of the antibodies of the present invention in relation to disease conditions. For example, the present invention is particularly useful but in no way limited to use in treating Plasmodium infections, their symptoms and pathologies.

Accordingly, another aspect of the present invention relates to a method of inhibiting, halting or delaying the onset or progression of a mammalian disease condition characterized by a Plasmodium infection said method comprising administering to said mammal an effective amount of an antibody as hereinbefore described.

Preferably said disease condition is malaria.

In yet another aspect the present invention relates to the use of an antibody in the manufacture of a medicament for inhibiting, halting or delaying the onset or progression of a disease condition characterized by the infection of a mammal by a Plasmodium.

Preferably said disease condition is malaria.

As detailed hereinbefore, Pf38, Pf12, Pf41, Pf92 and Pf113 correspond to previously unidentified molecules.

Accordingly, yet another aspect of the present invention is directed to an isolated protein as set forth in SEQ ID NO:2, 4, 6, 8 or 10 or having at least about 80% or greater identity to SEQ ID NO:2, 4, 6, 8 or 10 across the length of the sequence or a functional or immunogenic fragment or homologue, thereof.

Reference to “fragments” includes reference to parts and portions, from natural, synthetic or recombinant sources including fusion proteins. Parts or fragments include, for example, immunogenic regions.

A “homologue” refers to a sequence (nucleotide or protein) from another organism which exhibits at least about 80% and preferably 85%, 90%, 95%, 96%, 97%, 98% or 99% identity to the reference sequences. A preferred homologue is a Plasmodium homologue.

The term “protein” should be understood to encompass peptides, polypeptides and proteins. It should also be understood that these terms are used interchangeably herein. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

Preferably, said 80% or greater similarity is a reference to 85%, 90%, 91%, 92%, 93%, 94%, 95%, 96%, 97%, 98% or 99% similarity.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

As used herein, in terms of both the claimed proteins and nucleic acid molecules, the term “isolated” means that the material is removed from its original environment (e.g., the natural environment if it is naturally occurring). For example, a naturally occurring polynucleotide or protein present in a living animal is not isolated, but the same polynucleotide or protein, separated from some or all of the coexisting materials in the natural system, is isolated. Such polynucleotides could be part of a vector and/or such polynucleotides or protein could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

Proteins of the invention can be isolated from natural sources, be synthetic, or be recombinantly generated polypeptides. Peptides and proteins can be recombinantly expressed in vitro or in vivo. The proteins of the invention can be made and isolated using any method known in the art. Proteins of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

Proteins of the invention can also be synthesized and expressed as fusion proteins with one or more additional domains linked thereto for, e.g., to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising protein to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification while the enterokinase cleavage site provides a means for purifying a region from the remainder of the fusion protein. Technology pertaining to vectors encoding fusion proteins and application of fusion proteins are well described in the scientific and patent literature, see e.g., Kroll (1993) DNA Cell. Biol., 12:441-53.

Still another aspect of the present invention is directed to a protein encoded by a nucleotide sequence as set forth in SEQ ID NOs:1, 3, 5, 7 or 9 or the sequence complementary to a sequence capable of hybridizing to SEQ ID NOs:1, 3, 5, 7 or 9 under low stringency conditions and which encodes an amino acid sequence as set forth in SEQ ID NOs:2, 4, 6, 8 or 10 or having at least about 80% or greater identity to SEQ ID NOs:2, 4, 6, 8 or 10 across the length of the sequence.

Another aspect of the present invention is directed to an isolated nucleic acid selected from the list consisting of:

(i) An isolated nucleic acid molecule or functional fragment or homologue comprising a nucleotide sequence encoding, or complementary to a sequence encoding, an amino acid sequence substantially as set forth in SEQ ID NO:2, 4, 6, 8 or 10 or a functional or immunogenic fragment or homologue thereof, or an amino acid sequence having at least 80%, 90%, 95%, 96%, 97%, 98%, 99% or more sequence identity to SEQ ID NO:2, 4, 6, 8 or 10 over the length of the sequence, and/or a nucleic acid sequence capable of hybridizing to said nucleic acid molecule under low stringency conditions at 42° C.

(ii) An isolated nucleic acid molecule or functional or immunogenic fragment or homologue thereof comprising a nucleotide sequence encoding, or complementary to said sequence, wherein said nucleotide sequence is substantially as set forth in SEQ ID NO:1, 3, 5, 7 or 9 or a nucleotide sequence having at least about 80%, 90%, 95%, 96%, 97%, 98%, 99% or more identity over the length of the sequence of a nucleotide sequence capable of hybridizing to SEQ ID NO:1, 3, 5, 7 or 9 or complementary form thereof under low stringency conditions at 42° C.

(iii) An isolated nucleic acid molecule or derivative, homologue or analogue thereof comprising a nucleotide sequence as set forth in SEQ ID NO:1, 3, 5, 7 or 9.

The present invention should be understood to extend to the genomic DNA form of the cDNA nucleotide sequences detailed above.

The phrases “nucleic acid” or “nucleic acid sequence” as used herein refer to an oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

An “expression product” includes an RNA molecule such as an mRNA transcript as well as a protein. Some genes are non-protein encoding genes and produce mRNA or other RNA molecules and are involved in regulation by RNA:DNA, RNA:RNA or RNA:protein interaction. The RNA (e.g., mRNA) may act directly or via the induction of other molecules such as RNAi or via products mediated from splicing events (e.g., exons or introns). Short, interfering RNA (si-RNA) is also contemplated by the present invention. Other genes encode mRNA transcripts which are then translated into proteins. A protein includes a polypeptide. The differentially expressed nucleic acid molecules, therefore, may encode mRNAs only or, in addition, proteins. Both mRNAs and proteins are forms of “expression products”.

The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, parasitic, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which may be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids may be introduced into a genome or into the cytoplasm or a nucleus of a cell and expressed by a variety of conventional techniques, well described in the scientific and patent literature. See, e.g., Roberts (1987) Nature 328:731; Schneider (1995) Protein Expr. Purif. 6435:10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15:866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher (1992) J. Virol. 66:2731-2739; Johann (1992) J. Virol. 66:1635-1640). Adeno-associated virus (AAV)-based vectors can be used to transduce cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada (1996) Gene Ther. 3:957-964.

The term “expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The invention also provides a transformed cell comprising a nucleic acid sequence of the invention, e.g., a sequence encoding a polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. Exemplary parasitic cells include P. berghei. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

The term “similarity” as used herein includes exact identity between compared sequences at the nucleotide or amino acid level. Where there is non-identity at the nucleotide level, “similarity” includes differences between sequences which may encode different amino acids that are nevertheless related to each other at the structural, functional, biochemical and/or conformational levels. In a particularly preferred embodiment, nucleotide sequence comparisons are made at the level of identity rather than similarity.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, Chapter 15, 1994-1998). A range of other algorithms may be used to compare the nucleotide and amino acid sequences such as but not limited to PILEUP, CLUSTALW, SEQUENCHER or VectorNTI.

The terms “sequence similarity” and “sequence identity” as used herein refers to the extent that sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

As detailed above, and more specifically, protein and/or nucleic acid sequence identities (homologies) may be evaluated using any of the variety of sequence comparison algorithms and programs known in the art. The extent of sequence identity (homology) may be determined using any computer program and associated parameters, including those described herein, such as BLAST 2.2.2. or FASTA version 3.0t78, with the default parameters. For example, the sequence comparison algorithm is a BLAST version algorithm. In one aspect, for nucleic acid sequence identity analysis, the BLAST nucleotide parameters comprise word size=11, expect 10, filter low complexity with DUST, cost to open gap=5, cost to extend gap 2, penalty for mismatch=−3, reward for match=1, Dropoff (X) for BLAST extensions in bits=20, final X dropoff value for gapped alignment=50, and all other options are set to default. In one aspect, for polypeptide sequence identity analysis the sequence comparison algorithm is a BLAST version algorithm, e.g., where the BLAST nucleotide parameters comprise word size=3, expect=10, filter low complexity with SEG, cost to open gap=11, cost to extend gap=1, similarity matrix Blosum62, Dropoff (X) for blast extensions in bits=7, X dropoff value for gapped alignment (in bits)=15, final X dropoff value for gapped alignment=25.

Exemplary algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410,1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993). Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications.

BLAST, BLAST 2.0 and BLAST 2.2.2 algorithms are also used to practice the invention. They are described, e.g., in; Altschul, 1990, supra. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blast. There are about 38 setting options in the BLAST 2.2.2 program. In this exemplary aspect of the invention, all default values are used except for the default filtering setting (i.e., all parameters set to default except filtering which is set to OFF); in its place a “-F F” setting is used, which disables filtering. Use of default filtering often results in Karlin-Altschul violations due to short length of sequence.

The default values used in this exemplary aspect of the invention include:

“Filter for low complexity: ON

Word Size: 3

Matrix: Blosum62

Gap Costs Existence: 11

Extension: 1”

Other default settings are: filter for low complexity OFF, word size of 3 for protein, BLOSUM62 matrix, gap existence penalty of −11 and a gap extension penalty of −1.

The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention are compared to a reference sequence of the same number of contiguous positions after the two sequences are optimally aligned. If the reference sequence has the requisite sequence identity to an exemplary polypeptide or nucleic acid sequence of the invention, that sequence is within the scope of the invention.

The phrase “substantially identical” in the context of two nucleic acids or polypeptides, can refer to two or more sequences that have, e.g., at least about at least 90%, 91%, 92%, 93%, 94%, 95%, 96%, 97%, 98%, 99% or more sequence identity or more nucleotide or amino acid residue (sequence) identity, when compared and aligned for maximum correspondence, as measured using one any known sequence comparison algorithm, as discussed in detail below, or by visual inspection. In alternative aspects, the invention provides nucleic acid and polypeptide sequences having substantial identity to an exemplary sequence of the invention. Nucleic acid sequences of the invention can be substantially identical over the entire length of a polypeptide coding region.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

The invention provides isolated or recombinant nucleic acids that hybridize under stringent conditions to an exemplary sequence of the invention. In alternative aspects, the stringent conditions are highly stringent conditions, medium stringent conditions or low stringent conditions, as known in the art and as described herein. These methods may be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Hybridization” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % [Marmur and Doty, J. Mol. Biol. 5:109, 1962]. However, the Tₘ of a duplex DNA decreases by 1° C. with every increase of 1% in the number of mismatch base pairs [Bonner and Laskey, Eur. J. Biochem. 46: 83, 1974]. Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Where nucleic acids of the invention are defined by their ability to hybridize under high stringency, these conditions comprise about 50% formamide at about 37° C. to 42° C. In one aspect, nucleic acids of the invention are defined by their ability to hybridize under reduced stringency comprising conditions in about 35% to 25% formamide at about 30° C. to 35° C. Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions are well known in the art. Hybridization conditions are discussed further, below.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

In yet another aspect, the present invention extends to mutants of Pf38, Pf12, Pf41, Pf92 or Pf113. Reference to “mutation” and “mutant” should be understood as a reference to any change, alteration or other modification, whether occurring naturally or non-naturally, which results in a molecule exhibiting functionally altered activity relative the activity of the wild-type molecule.

As detailed hereinbefore, still another aspect of the present invention is directed to antibodies to the protein and nucleic acid molecules and mutants herein defined. Such antibodies may be monoclonal or polyclonal and may be selected from naturally occurring antibodies or may be specifically raised. The polypeptide or nucleic acid antigen may first need to be associated with a carrier molecule to achieve immunogenicity. An antibody “to” a molecule includes an antibody specific for said molecule.

These antibodies can be used to isolate, identify or quantify a polypeptide of the invention or related polypeptides.

The term “antibody” includes a peptide or polypeptide derived from, modeled after or substantially encoded by an immunoglobulin gene or immunoglobulin genes, or fragments thereof, capable of specifically binding an antigen or epitope, see, e.g., Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Antibodies to the molecules of the present invention may be monoclonal or polyclonal and may be selected from naturally occurring antibodies or may be specifically raised to these polypeptide and gene products. The present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying the subject polypeptide or nucleic acid molecule.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

The present invention is further described by reference to the following non-limiting examples.

### EXAMPLE 1

### Antigen Identification in Plasmodium Falciparum Blood-Stages by Membrane Proteomics

Materials and Methods

Preparation of Detergent Resistant Membranes

Sorbitol synchronized parasite cultures at 8-10% parasitaemia were pelleted (1500 rpm Beckman GS-6 centrifuge) and late stage schizonts (approximately 44 hours post-invasion) were purified using Miltenyi Biotec Vario MACS CS magnetic separation columns. Metabolic labeling of GPI anchored proteins was achieved by incubating parasite-infected erythrocytes with 10 μCi/ml of D-[6-³H(N)]glucosamine hydrochloride in glucose-free RPMI medium supplemented with 10 mM fructose, 25 mM Hepes, 0.2 mM hypoxanthine at 37° C. for 4 hours. Parasites were washed twice in culture medium prior to saponin lysis (0.15% saponin, 10 min, on ice). Samples were pelleted at 2800 rpm for 10 mins (Beckman GS-6 centrifuge) and washed three times in MES-buffered saline (25 mM MES (Sigma) pH 6.5, 150 mM NaCl). Parasites were resuspended to a volume of 1.5 mL in MES containing a Roche Complete protease inhibitor cocktail tablet and chilled to 0° C. on an ice water slurry. Parasite samples were cooled to 0° C. and an equal volume of chilled (0° C.) 1% Triton X-100 (SigmaUltra) in MES at 0° C. was added to give a final concentration of 0.5% TX-100. Samples maintained at 0° C. for 30 mins (3 mL total) were resuspended every 10 mins, then centrifuged at 30,000 rpm (2° C. for 30 mins) in a pre-chilled TLA 100.3 rotor in a Beckman Optima MAX-E Ultracentrifuge. The supernatant was and the pellet suspended in 184 μL 0.5% Triton X-100 in MES buffer containing protease inhibitors to which an equal volume of 80% sucrose (ultra pure BRL Bethesda Research Laboratories, Inc.) in MES buffer was added. The pellet material (368 μL total, 40% sucrose) was transferred to a 2.2 mL open top thin-walled polyallomer tube and overlayed with 1.1 mL 35% sucrose in MES buffer, followed by 733 μL 5% sucrose in MES buffer to form a sucrose step gradient (all solutions at 0° C.). The gradient was centrifuged at 55,000 rpm for 18 hrs (2° C.) with low acceleration and no deceleration in a pre-chilled Beckman TLS-55 swing rotor. Following ultra centrifugation 15 equal fractions (146 μL) were removed from the top of the gradient, snap frozen on dry ice and stored at −80° C.

For proteomic analysis, the 15 sucrose gradient fractions were pooled according to the distribution pattern of MSP-1₂₀₀ (determined by Western blotting), such that Fractions 1-4 (representing the top of the gradient) formed Pool 1, Fractions 5-9 formed Pool 2, Fractions 10-13 formed Pool 3 and Fractions 14-15 formed Pool 4 (pellet material). Pooled fractions were diluted to 3 mL with HT-PBS at 0° C. and centrifuged at 30,000 rpm for 1 hr (2° C.) in TLA 100.3 rotor in a Beckman Optima MAX-E Ultracentrifuge. The supernatant was discarded and an additional 3 mL HT-PBS (0° C.) carefully added to the tubes without disruption of the pellet material and re-centrifuged as above. Supernatant was discarded and the pellets were snap frozen on dry ice for proteomic analysis.

Immunodetection of Detergent Resistant Membrane Proteins.

DRM fractions from the sucrose gradient were resuspended in an equal volume of 2× non-reducing sample buffer and placed at 70° C. for 10 mins. Samples (20 μL) were loaded onto a 4-20% gradient Gradipore Long-Life TRIS-HEPES-SDS pre-cast polyacrylamide mini gel, spiking the first fraction with 6 ILL Bio-Rad pre-stained Precision Plus Protein Standards. Protein samples were electrophoresed under non-reducing conditions and transferred to Immobilon-P transfer membranes (Millipore) for Western blotting as described [O'Donnell et al., 2001, J. Exp. Med. 193, 1403-1412].

Protein Digestion and MudPIT Analysis.

Each membrane/protein pellet was resuspend in 20 μl of 90% formic acid containing 500 mg/ml cyanogen bromide, and incubate overnight at room temperature (RT) protected from light. Two volumes (40 μl) of 30% NH₄OH was added drop by drop, followed by the addition of three volumes (180 μl) of saturated NH₄HCO₃ drop by drop. The preceding steps are performed under a hood to prevent exposure to cyanogen bromide. After verifying that the pH of each solution is ˜8.5, solid urea is added to a final concentration of 8M. Disulfide bonds are reduced by adding TCEP to 5 mM and incubated at RT for 30 min. Cysteines are alkylated by adding iodoacetamide to 20 mM and incubating at RT for 30 min. protected from light. Sequencing grade endoproteinase Lys-C (Roche) was then added at an estimated ratio of 1:100 (enzyme:substrate, wt/wt) and incubate at 37° C. for ˜16 hrs. The solution was then diluted two fold by adding an equal volume (345 μl) of 100 mM Tris pH 8.5, and add CaCl₂ to 2 mM. Sequencing grade modified trypsin (Promega) was then added at an estimated ratio of 1:100 and incubated at 37° C. for ˜16 hrs. 90% formic acid was then added to a final concentration of 4%.

Each sample was essentially loaded on a “3-phase MudPIT column” and developed with six elution steps using an HP1100 HPLC (Hewlett Packard) online with an LCQ Deca ion trap mass spectrometer (Thermo) as previously described [McDonald et al., 2002, Dis. Markers 18:99-105]. Data dependent MS/MS spectra were collected as previously described [Washburn et al., 2001, Nat. Biotechnol. 19:242-247]. MS/MS spectra were searched against a P. falciparum protein database (Oct. 3, 2002 release, with the manually added sequence of RAMA) combined with human, mouse and rat databases using the search algorithm SEQUEST. Peptides identifications were filtered using default settings (except where noted) using the program DTASelect, and samples were compared using the program Contrast [Tabb et al., 2002, J. Proteome Res. 1:21-26].

Live Fluorescent Imaging.

GFP-fusion proteins for localization studies were encoded in transfection constructs under the regulation the tetracycline inducible expression system [Meissner et al., 2005, supra] Anhydrotetracycline was removed from parasite cultures 72 hours prior to live imaging (in the presence of 2.5 nM WR99210) to allow expression of the GFP-fusion. Prior to microscopy parasites were incubated in culture medium containing 100 ng/ml DAPI. Late blood-stage parasite fluorescence images were captured using a Carl Zeiss Axioskop microscope, with a PCO SensiCam and Axiovision 2 software.

Immunofluorescence Assays

Immunofluorescence assays, blood-stage parasites were fixed as described previously [Tonkin et al., 2004, Mol. Biochem. Parasitol, 137: 13-21] and then dried onto poly-L-lysine-coated slides. Parasites were blocked and permeabilized in 1% bovine serum albumin and 0.2% Triton X-100 in PBS and then probed with mouse anti-Pf41 serum (1:100) and rabbit anti-MSP-1₁₉ IgG [de koning-Ward et al., 2003, J. Exp. Med., 198: 869-875] (0.02 mg/ml). Polyclonal mouse sera were raised to a recombinant MBP-fusion protein containing a 115-amino acid spacer region between the two Cys₆ domains of Pf41 and a C-terminal 6× Itis tag. The fusion protein was expressed in BL-21 Escherichia coli cells and purified over nickel resin (Qiagen). Bound antibodies were then visualized with Alexa Fluor 568 nm anti-rabbit IgG and Alex Fluor 488 nm anti-mouse IgG (Molecular Probes) diluted 1:1000. Parasites were mounted in Vectashield containing 4′,6-diamindino-2-phenylindole (Vecta Laboratories).

Immunoprecipitation of GFP Fusion Proteins

Immunoprecipiation of GFP fusion proteins expressed in P. falciparum, anhydrotetracycline was removed from parasite cultures 72 h prior to solubilization to induce expression of the transgene. Schizonts were solubilized in 1% Triton X-100, PBS, pH 7.6, at room temperature for 30 min in the presence of Roche Applied Science Complete protease inhibitor mixture. Samples were preabsorbed with control (from individuals not exposed to malaria) IgG-coated Sepharose (100 μl of a 50% suspension, 2 h, 4° C.) and then incubated with Pool M human IgG-coated Sepharose (from individuals not exposed to malaria) beads (100 μl of a 50% suspension) and incubated at 4° C. for 4 h. Pool M was prepared from pooled sera obtained from highly exposed (P. falciparum immune) Papua New Guinean adults [O'Donnell et al., 2001, J. Exp. Med., 193: 1403-1412]. The unbound fraction (˜1 ml) was retained, and beads were washed 3×10 min in immunoprecipitation buffer (PBS, pH 7.6, containing 1% Triton X-100 and protease inhibition). Bound material was eluted with the addition of an equal bed volume of non-reducing SDS sample buffer (on ice, 5 min) followed by the subsequent addition of an equal bed volume of non-reducing sample buffer at 70° C. for 10 min.

P. falciparum Culture and Transfection.

P. falciparum 3D7 strain parasites were cultured and synchronized using standard procedures [Lambros et al., 1979, J. Parasitol. 65:418-420; Trager et al., 1976, Science 193:673-675]. Ring-stage parasites (˜1% parasitemia) were transfected with 100 μg of purified plasmid DNA (Plasmid Maxi Kit, Qiagen) as described previously [Crabb et al., 1996, Proc. Natl. Acad. Sci. U.S.A. 93:7289-7294], using modified electroporation conditions [Fidock et al., 1997, Proc. Natl. Acad. Sci. U.S.A. 94:10931-10936].

Results

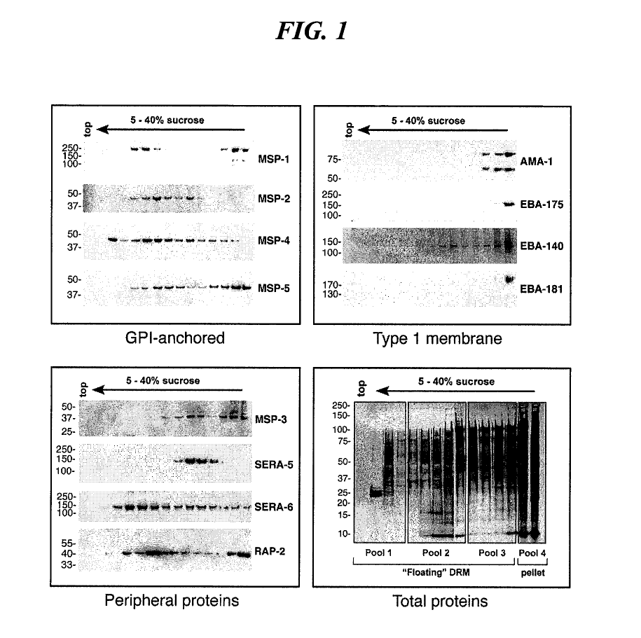

Plasma membrane proteins of many eukaryotes, including P. falciparum [Wang et al., 2003, Mol. Biochem. Parasitol. 130:149-153], appear to be enriched in detergent-resistant membrane (DRM) domains. To further characterize the surface coat of the bloodstream form of P. falciparum parasites the proteome of DRMs in this life stage was determined. Sucrose gradient flotation was used to purify DRMs from schizonts; an intraerythrocytic stage that consists of maturing merozoites that are enclosed in a parasitophorous vacuole (PV). The effectiveness of this approach was examined by western blot analysis of individual fractions (FIG. 1). Each of the GPI-anchored proteins examined (MSP-1, -2, -4 and -5) was recovered in the buoyant fractions, toward the centre and top of the gradient. These proteins were well separated from the single-pass type 1 integral membrane proteins, which remained in the bottom fractions. It was evident that peripheral proteins located in the PV or rhoptry organelles also tended to associate with DRMs (FIG. 1). These proteins each possess a signal sequence but do not have an obvious membrane anchor element. It is presumed that in some if not most instances the association of these proteins with DRMs results from an interaction with a GPI-anchored protein. It should be noted that unlike all other proteins examined, the SERA proteins were only detected in whole infected erythrocytes that had not been pre-treated with saponin, a process that removes much of the surrounding erythrocyte proteins.

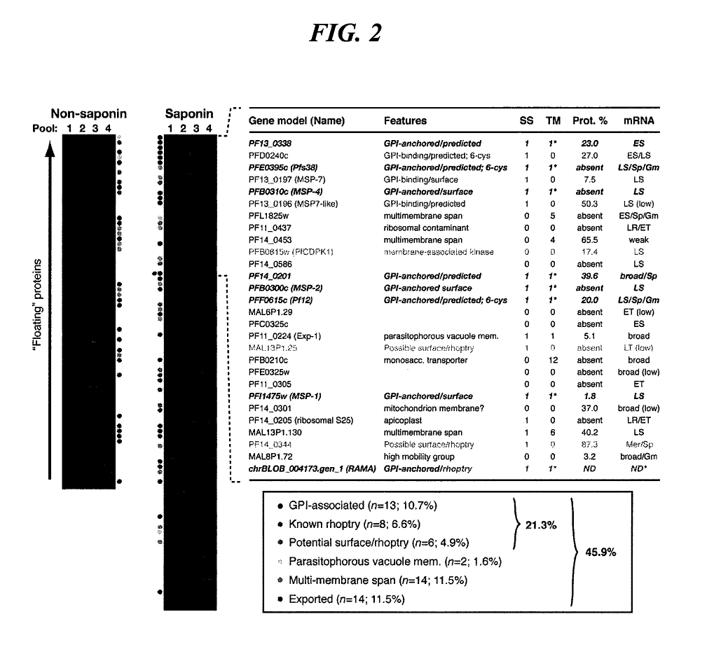

To analyze the proteome of schizont-stage DRMs, DRM fractions from both saponin and non-saponin lysed parasites were combined into 4 pools (FIG. 1) and the proteome of washed membranes present in each pool determined by MudPIT [Washburn et al. 2001, supra]. Ninety-four and 70 parasite proteins were detected in the saponin and non-saponin DRM proteomes, respectively (FIG. 2 and Tables 1 and 2). There was strong overlap between proteomes with 42/70 (60%) of the non-saponin DRM proteins also found in the saponin DRM proteome; hence a total of 122 DRM proteins were detected. Only high confidence proteins were included in these lists, criteria that included the detection of at least two different peptides for each protein. Proteins were ordered according to the degree of buoyancy in their respective sucrose gradients such that the DRM proteins primarily found in the highest floating fractions are present toward the top of the list (FIG. 2).

Overall, both the saponin- and non-saponin-treated schizont DRM proteomes were greatly enriched for proteins predicted to be membrane-associated by means of an encoded signal sequence and/or a transmembrane domain; from 24% in the whole schizont proteome [Le Roch et al., 2004, supra] to 65-73% in the DRM proteomes. Four classes of protein were particularly prominent in DRM fractions: GPI-associated proteins (defined below), rhoptry proteins, multi-membrane spanning proteins and proteins predicted to be exported from the parasite into the host erythrocyte cytosol [Marti et al., 2004, Science 306:1930-1933]. Proteins in these groups were found mostly in floating fractions (Pools 1-3) and collectively they comprise almost half of the 122 DRM proteins detected (FIG. 2).

Analogous to proteomic analyses of mammalian “lipid rafts” [Foster et al., 2003, Proc. Natl. Acad. Sci. U.S.A. 100:5813-5818], the fractionation procedure appears to have enriched for lipid-anchored membrane proteins—with a strong peptide coverage of GPI-associated proteins in the highest floating DRM fractions (FIG. 2). Good peptide coverage was obtained for known abundant GPI-anchored proteins such as MSP-2 and MSP-4, peptides of which were surprisingly poorly represented, or absent altogether, in various whole schizont P. falciparum proteomes [Lasonder et al., Nature 419:537-542, 2002; Florens et al., 2002, Nature 419:520-526; Le Roch et al., 2004, supra]. Hence, the extraction procedures used to obtain DRMs has facilitated the identification of a subset of relatively abundant proteins that are not effectively solubilized in the whole cell extracts.

Thirteen GPI-associated proteins were detected which can be categorized as follows: (1) known GPI-anchored proteins, (2) predicted GPI-anchored proteins (i.e., those possessing an N-terminal signal sequence and a characteristic C-terminal hydrophobic domain), (3) proteins that bind to GPI-anchored proteins (MSP-7; [Pachebat et al., 2001, Mol. Biochem. Parasitol. 117:83-89]), and (4) predicted proteins that bind to GPI-anchored proteins (i.e., protein homologues of groups 2 or 3 and the SERAs). Most GPI-associated proteins detected in the non-saponin extract were also present in the saponin DRM proteome; only SERA5 and SERA6 were unique to the non-saponin list as expected from FIG. 1.

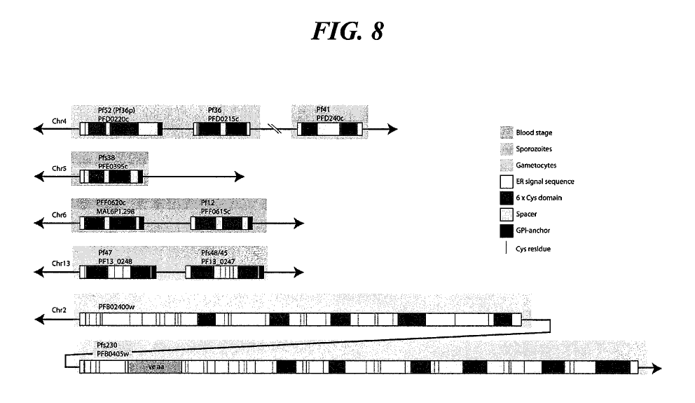

Four of the 6 known GPI-anchored merozoite proteins (MSP-1, -2, -4 and RAMA) were in the DRM proteomes and each was prominent in high floating fractions. The absence of MSP-10 from the list was expected, as this protein does not appear to incorporate into DRMs [Wang et al., 2003, supra; Glison et al., 2006, supra]. It is less clear why MSP-5 was not detected although the western blot in FIG. 1 suggests that only a small proportion of MSP-5 is present in buoyant DRM fractions. Importantly, each of the 4 predicted GPI-anchored proteins were prominent in both saponin and non-saponin extracts. Two of these, encoded by PFE0395c and PFF0615c, are comprised almost entirely of dual “six-cysteine” (6-cys) domains, a fold found in a family of Plasmodium proteins, some of which localize to the surface of gametocyte stage parasites and have potential as transmission blocking vaccines [Carter, R. (2001), Vaccine 19:2309-2314] (FIG. 8). Gene knockout experiments have confirmed a key role for the best-characterized member of this family, Pfs48/45, in gamete fertilization, seemingly as an adhesive surface protein [van Dijk et al., 2001, Cell 104:153-164]. Although not known to be blood-stage proteins, genes encoding PFE0395c or PFF0615c have been recognized previously and were termed Pfs38 (renamed Pf38 here) and Pf12 respectively [Templeton et al., 1999, Mol. Biochem. Parasitol. 101:223-227; Elliott et al., 1990, Proc. Natl. Acad. Sci. U.S.A. 87:6363-6367; Thompson et al., 2001, Mol. Biochem. Parasitol. 118:147-154]. The two other putative GPI-anchored proteins detected, PF13_{—}0338 and PF14_{—}0201 (termed Pf92 and Pf113 after their predicted molecular weights), are large novel proteins that do not possess obvious homology to other proteins, although both possess cysteine-rich domains.

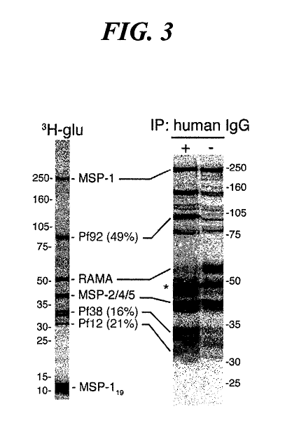

To further validate the presence of these novel GPI-anchored proteins, infected erythrocytes were metabolically labeled with 3H-glucosamine (only GPI-anchors incorporate this sugar in Plasmodium parasites) and the identity of labeled bands determined by LC-MS/MS (FIG. 3). This approach confirmed the presence of the newly identified GPI-anchored proteins Pf92, Pf38 and Pf12, each of which appear to be relatively abundant based on the strength of signal relative to MSP-1 and MSP-2. An extensive analysis of the 3H-glucosamine species [Glison et al., (2006), supra], has revealed that several GPI-anchored species remained unassigned, one of which, a relatively minor species at ˜130 kDa, is close to the predicted molecular weight of Pf113. Importantly, each of Pf92, Pf38 and Pf12 appear to react well with antibodies present in individuals naturally exposed to P. falciparum while some other GPI-anchored proteins (e.g., RAMA) reacted poorly (FIG. 3). Although not previously defined as a blood-stage protein, such reactivity with human antibodies was already known for a recombinant form of Pf12 expressed on the surface of mammalian cells [Elliott et al., 1990, supra]. Together, this data highlights the surface exposure (and antigenic potential most of the newly identified GPI-anchored proteins.

PFD0240c (Pf41) represents another dual 6-cys protein that segregates into the DRM fraction. As with some other members of this family (e.g., Pfs230), Pf41 does not encode a C-terminal GPI-attachment sequence (FIG. 8). A recombinant form of Pf41 reacts strongly with human anti-P. falciparum antibodies consistent with a designation as a surface-exposed antigen (FIG. 3). MSP7-like protein (PF13_{—}0196), like MSP-7, is a peripheral protein that presumably associates with DRMs by virtue of an interaction with MSP-1 [Pachebat et al., 2001, supra]. Several other probable peripheral proteins (i.e., those with a signal sequence in the absence of other known organellar targeting elements) were detected some of which potentially represent proteins that associate with GPI-anchored species (orange dots in FIG. 2). While a number of putative GPI-binding proteins were identified, several well known, and apparently abundant, peripheral merozoite surface proteins were not detected in either DRM proteome. For example, members of the chromosome 10 MSP-3/-6 family were absent from DRMs, suggesting that these proteins are unlikely to interact strongly with GPI-anchored surface proteins.

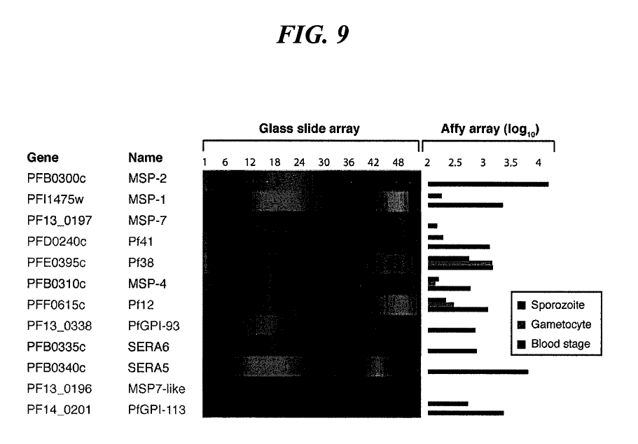

Genes encoding most of the 13 GPI-associated proteins are transcribed in blood-stages, with maximal levels of expression late in the blood-stage cycle (FIG. 2 and FIG. 9; [Bozdech et al., 2003, supra; Le Roch et al., 2003, supra]. The exception is the novel putative GPI-anchored protein PF14_{—}0201, which has a much broader blood-stage profile and is maximally transcribed earlier in the life cycle.

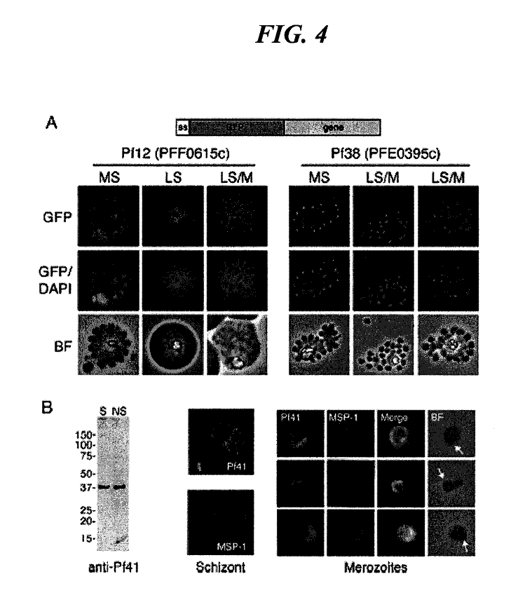

To investigate the localization of two newly identified 6-cys proteins (Pf38 and Pf12) a green fluorescent protein (GFP) targeting approach was employed using an inducible expression system that directs strong, schizont-stage expression of transgenes [Meissner et al., 2005, supra]. These two GPI-anchored proteins were localized to the merozoite surface using this approach (FIG. 4A). Interestingly, Pf38 also localized to the apical region of the parasite, exclusively at an early stage of schizont development.

With respect to rhoptry proteins, the GPI-anchored rhoptry protein RAMA [Topolska et al., 2004, supra], was detected in the DRM proteome (FIG. 2). In addition, all members of the high (RhopH) and low (RAP) protein complexes were identified (FIG. 2). It is likely that one or both of these complexes interact with a rhoptry resident GPI-anchored protein. Indeed, fluorescence resonance energy transfer analysis has suggested that both could be interacting with RAMA [Topolska et al., 2004, supra].

The resident PV membrane proteins EXP-1 and -2 were prominent in the DRM proteomes, especially in untreated schizonts, consistent with the previously described presence of EXP-1 in lipid raft-like structures in this membrane [Lauer et al., 2000, EMBO J. 19:3556-3564]. Also of note is PfGAP50 the homolog of which in Toxoplasma gondii is associated with the inner membrane complex and is considered a key component of the myosin motor system that drives gliding motility and invasion [Gaskins et al., 2004, J. Cell Biol. 165:383-393]. As expected, several multi-membrane spanning proteins were also detected in the DRM proteome including some transporters and ATPases. Perhaps the most interesting in terms of a likely surface or apical organelle location are the hypothetical proteins encoded by PFL1825w and MAL13P1.130 (FIG. 2). Both genes are transcribed most strongly late in blood-stage development. In other cells, acylated proteins are also commonly found in DRMs and indeed one such protein, CDPK1 (PFBO815w) [Moskes et al., 2004, Mol. Microbiol. 54:676-691], was amongst the prominent floating proteins. Other similarly modified membrane proteins are potentially amongst those DRM proteins that do not possess an obvious signal sequence or transmembrane domain(s).

To investigate the localization of Pf41, antibodies were raised to the unusually large interdomain region of this protein. The antibodies recognized an ˜40-kDa saponin-resistant protein that is spread over the parasite surface in whole schizonts but is concentrated toward the apical end in free merozoites (FIG. 4B).

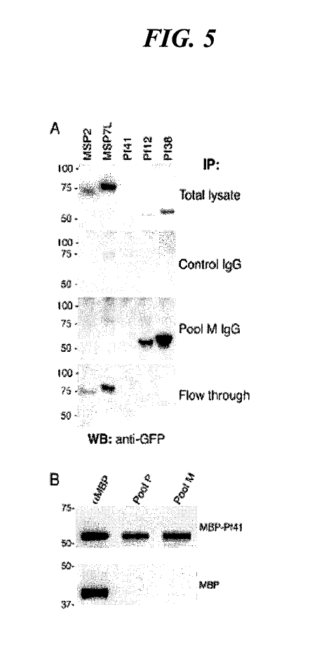

Pf12 and Pf38 fusion proteins expressed in P. falciparum were strongly recognized by antibodies present in individuals naturally exposed to P. falciparum (FIG. 5A). Furthermore, an E. coli-expressed version of the central region of Pf41 also bound to antibodies present in human immune sera (FIG. 5B).

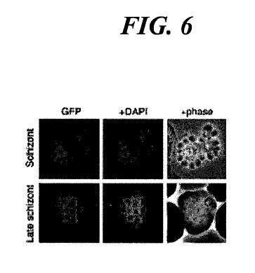

Using inducible GFP expression system, it was shown that another predicted GPI-anchored protein, Pf92, also localized to the merozoite surface (FIG. 6). Transcription of the gene encoding Pf92 was also co-regulated with MSP-2 (FIG. 9). This large protein includes 14 cysteine residues that are relatively evenly spaced throughout the entire molecule.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1

| | | SS + | | | | | | | |
|---|---|---|---|---|---|---|---|---|---|
| Gene | Name | NS* | POOL 1 | POOL 2 | POOL 3 | POOL 4 | SS | TMD | Features |
| PF13_0338 | hypothetical protein | + | 6.3** | 14.4 | 2.4 | 0 | 1 | 1 | GPI-anchorod (predicted) |
| PFD0240c | hypothetical protein | | 4 | 14.8 | 0 | 0 | 1 | 0 | GPI-associated (predicted; 6-cys) |
| PFE0395c | hypothetical protein | + | 3.2 | 28.4 | 3.4 | 0 | 1 | 1 | GPI-anchorod (PtS38; 6-cys) |
| PF13_0197 | merozoite surface protein 7 | | 0 | 34.5 | 0 | 3.1 | 1 | 0 | GPI-associated surface |
| PFB0310c | merozoite surface protein 4 | | 0 | 12.5 | 0 | 0 | 1 | 1 | GPI-anchorod surface |
| PF13_0196 | MSP7-like protein | | 0 | 15.8 | 0 | 0 | 1 | 0 | GPI-associated (predicted) |
| PFL1825w | hypothetical protein | | 0 | 11.9 | 0 | 0 | 0 | 5 | late stage exp |
| PF11_0437 | hypothetical protein | | 0 | 15.7 | 0 | 0 | 0 | 0 | ribosomal |
| PF14_0453 | hypothetical protein | | 0 | 6.1 | 0 | 0 | 0 | 4 | |
| PFB0815w | protein kinase (PICDPK1) | | 0 | 11.1 | 0 | 0 | 0 | 0 | acylated, poss surface protein |
| PF14_0586 | hypothetical protein | | 0 | 8 | 0 | 0 | 0 | 0 | Mem assoc. MORN repeats/ |
| | | | | | | | | | PI kinase/Late stage |
| PF14_0201 | hypothetical protein | + | 0 | 7.8 | 0 | 1.8 | 1 | 1 | GPI-anchorod (predicted) |
| PFB0300c | merozoite surface protein 2 | + | 35.3 | 36 | 34.2 | 20.6 | 1 | 1 | GPI-anchorod surface |
| MAL6P1.299 | pf12 | + | 3.7 | 3.7 | 0 | 0 | 1 | 1 | GPI-anchorod (predicted; 6-cys) |
| MAL6P1.29 | hypothetical protein | | 1.2 | 1.2 | 0 | 0 | 0 | 0 | |
| PFC0325c | hypothetical protein | | 0.5 | 0.7 | 0 | 0 | 0 | 0 | |
| PF11_0224 | exported protein 1 (exp-2) | + | 0 | 7.4 | 19.1 | 0 | 1 | 1 | PV membrane |
| MAL13P1.25 | hypothetical protein | | 0 | 0.5 | 0.8 | 0 | 1 | 0 | Poss Rhoptry/surface protein |
| PFB0210c | monosaccharide transporter | | 0 | 3.4 | 3.4 | 0 | 0 | 12 | transporter |
| PFE0325w | hypothetical protein | | 1.2 | 0 | 0 | 0 | 0 | 0 | |
| PF11_0305 | hypothetical protein | | 3.5 | 0 | 0 | 3.5 | 0 | 0 | Mcp2/Sun (Fmu) family |
| PF11475w | merozoite surface protein 1 | + | 0.4 | 36.2 | 12.3 | 18.2 | 1 | 1 | GPI-anchorod surface |
| PF14_0301 | hypothetical protein | | 0 | 9.3 | 3.1 | 3.1 | 0 | 0 | mitochondrion membrane? |
| PF14_0205 | ribosomal protein S25 | + | 0 | 17.0 | 8.9 | 8.9 | 1 | 0 | potential apicoplast |
| MAL13P1.130 | hypothetical protein | + | 0 | 7.6 | 3.6 | 5 | 1 | 6 | late stage exp |
| PF14_0344 | hypothetical protein | + | 0 | 5.4 | 3.4 | 4.1 | 1 | 0 | Poss Rhoptry/surface protein |
| MAL8P1.72 | high mobility group protein | + | 0 | 27.3 | 0 | 19.2 | 0 | 0 | probable contaminant |
| c1\|37220738\|cb | RAMA | | [symbol] | [symbol] | [symbol] | [symbol] | [symbol] | [symbol] | [symbol] |
| PF11780w | hypothetical protein | + | 0 | 5.7 | 0 | 6 | 1 | 0 | exported protein (RLE motif) |
| PF13_0252 | nucleoside transporter 1 | | 0 | 3.8 | 1.9 | 3.8 | 1 | 10 | transporter |
| PFE0060w | hypothetical protein | | 0 | 3.7 | 2.7 | 5.1 | 1 | 1 | exported protein (RLE motif) |
| PFI0880c | acid phosphatase (PIGAP50) | + | 0 | 9.3 | 4 | 12.4 | 1 | 1 | inner membrane complex |
| PF11_0175 | heat shock protein 101 | + | 0 | 10.7 | 5.6 | 16.4 | 1 | 0 | potential apicoplast/c [symbol] |
| PF10_0323 | hypothetical protein | | 0 | 7.6 | 6.2 | 14.1 | 1 | 1 | [symbol] |
| PFC0725c | transporter | + | 0 | 2.9 | 2.9 | 6.5 | 0 | 7 | transporter |
| PFE1600w | hypothetical protein | | 0 | 5.5 | 2.9 | 10.6 | 0 | 1 | exported protein (RLE motif) |
| MAL7P1.146 | hypothetical protein | + | 0 | 0.3 | 0 | 0.4 | 0 | 8 | |
| PF13_0143 | haxphoribosylpyrophosphate synthetase | | 0 | 3 | 0 | 3.9 | 0 | 1 | |
| MAL6P1.256 | mitochondrial import receptor | | 0 | 9.8 | 0 | 12 | 0 | 0 | |
| | subunit tom40 | | | | | | | | |
| PFD1170c | hypothetical protein (RESA-like) | + | 0 | 6.8 | 0 | 9.7 | 0 | 1 | exported protein (RLE motif) |
| PF13_0304 | elongation factor 1 alpha | + | 0 | 7.7 | 0 | 12.6 | 0 | 0 | |
| PF13_0305 | elongation factor 1 alpha | + | 0 | 7.7 | 0 | 12.6 | 0 | 0 | |
| PF11_0317 | structure maintenance of chromosome | | 0 | 0.4 | 0 | 0.9 | 0 | 0 | |
| | protein | | | | | | | | |
| PFL2215w | actin | + | 0 | 6.1 | 0 | 12 | 0 | 1 | |
| PFD0080c | hypothetical protein | + | 0 | 0 | 4.6 | 2.7 | 1 | 1 | exported protein (RLE motif) |
| MAL7P1.149 | hypothetical protein | | 0 | 0 | 0.9 | 0.6 | 1 | 0 | potential apicoplast |
| PFL2505c | hypothetical protein | | 0 | 0 | 0 | 1 | 1 | 7 | Poss Rhoptry/surface protein |
| [symbol] | [symbol] | [symbol] | 0 | 10.9 | [symbol] | [symbol] | [symbol] | [symbol] | [symbol] |
| [symbol] | [symbol] | [symbol] | 0 | [symbol] | [symbol] | [symbol] | [symbol] | [symbol] | [symbol] |
| PFD8_0054 | heat shock 70 kDa protein | + | 1.0 | 4.3 | 8.7 | 10.8 | 0 | 0 | |
| [symbol] | [symbol] | [symbol] | [symbol] | [symbol] | [symbol] | [symbol] | [symbol] | [symbol] | [symbol] |
| PF07_0128 | EBA-175 | | 0 | 0 | 0.7 | 0.8 | 1 | 1 | micronome |
| PF14_0615 | ATP synthase (C/AC39) subunit | + | 0 | 0 | 6.3 | 7.3 | 0 | 0 | |
| PF10_0144 | prohibitin (band7/stomatin-like) | | 0 | 0 | 2 | 3.9 | 0 | 1 | Poss Rhoptry/protein |
| [symbol] | [symbol] | [symbol] | [symbol] | [symbol] | [symbol] | [symbol] | [symbol] | [symbol] | [symbol] |
| PF08_0112 | hypothetical protein | | 0 | 0 | 2.8 | 5.3 | 0 | 0 | |
| PF07_0007 | hypothetical protein | | 0 | 0 | 13 | 20.3 | 0 | 1 | poss. exported protein (RLE motif) |
| PF11_0507 | antigen 332 | + | 0 | 0 | 0.4 | 2.1 | 0 | 0 | exported protein |
| PFD0090c | hypothetical protein | + | 0 | 0 | 2.6 | 6.8 | 0 | 0 | exported protein |
| [symbol] | [symbol] | [symbol] | [symbol] | [symbol] | [symbol] | [symbol] | [symbol] | [symbol] | [symbol] |
| [symbol] | [symbol] | [symbol] | 0 | 0 | [symbol] | [symbol] | [symbol] | [symbol] | [symbol] |
| PFI1625c | organic processing peptidase | | 0 | 0 | 3.3 | 9.3 | 0 | 0 | |
| PFL0590c | p-type ATPase | | 0 | 1.2 | 1.9 | 6.6 | 0 | 8 | |
| PF07_0008 | hypothetical protein | | 0 | 5.1 | 7.9 | 25.7 | 1 | 0 | poss. exported protein (RLE motif) |
| PFD0310w | 16 kDa sexual stage-specific protein | + | 7.6 | 7.6 | 7.6 | 32.5 | 1 | 1 | probable gametocyte contaminant |
| | precursor (Pfs16 | | | | | | | | |
| [symbol] | [symbol] | [symbol] | [symbol] | [symbol] | [symbol] | [symbol] | [symbol] | [symbol] | [symbol] |
| [symbol] | [symbol] | [symbol] | 0 | 0 | 0 | [symbol] | [symbol] | [symbol] | [symbol] |
| PF14_0553 | trophocite cysteine proteinase | | 0 | 0 | 0 | 3.7 | 1 | 0 | exported protein (RLE motif) |
| PFE1150w | multidrug resistance protein | + | 0 | 0 | 0 | 6 | 0 | 10 | |
| PF13_0065 | vacuolar ATP synthase catalytic | | 0 | 0 | 0 | 5.9 | 0 | 0 | |
| | subunit a | | | | | | | | |
| PFE0065w | selection binding protein (sbp1) | | 0 | 0 | 0 | 14.5 | 0 | 2 | exported protein |
| PF13_0213 | 60S ribosomal subunit protein L6e | | 0 | 0 | 0 | 14 | 0 | 0 | |
| PF14_0655 | RNA helicase-1 | | 0 | 0 | 0 | 6.5 | 0 | 0 | |
| PFD0305c | vacuolar ATP synthase subunit b | | 0 | 0 | 0 | 6.7 | 0 | 0 | |
| PF07_0070 | 60S ribosomal protein L11a | | 0 | 0 | 0 | 12.7 | 0 | 0 | |
| PF11_0313 | ribosomal phosphoprotein P0 | + | 0 | 0 | 0 | 13 | 0 | 0 | |
| PFE1155c | mitochondrial processing peptidase | | 0 | 0 | 0 | 7.1 | 0 | 1 | |
| | alpha subunit | | | | | | | | |
| PFE0040c | Mature parasite infected erythrocyte | + | 0 | 0 | 0 | 3.6 | 1 | 0 | exported protein (RLE motif) |
| | surface antigen (MESA) or PIEMP2 | | | | | | | | |
| PFI0875w | Heat shock protein | + | 0 | 0 | 0 | 6.1 | 1 | 0 | |
| PF08_0113 | vacuolar protein [symbol] ATPase subunit A | | 0 | 0 | 0 | 6.3 | 0 | 6 | |
| PF14_0578 | exported protein 2 (exp-2) | + | 0 | 0 | 0 | 8.4 | 1 | 0 | PV membrane |
| PF08_0137 | hypothetical protein | + | 0 | 0 | 0 | 9.9 | 1 | 1 | poss. exported protein (RLE motif) |
| PFE0965c | vacuolar ATP synthase | | 0 | 0 | 0 | 11.5 | 0 | 4 | |
| PF13_0228 | 40S ribosomal subunit protein S6 | + | 0 | 0 | 0 | 12.1 | 0 | 0 | |
| PF11_0351 | heat shock protein hsp70 homologue | | 0 | 0 | 0 | 7.8 | 0 | 0 | |
| PF13_0346 | ubiquitin/ribosomal fusion protein | + | 0 | 0 | 0 | 22.7 | 0 | 0 | |
| | ub052 homologue | | | | | | | | |
| PF10_0817 | ribosomal protein L30c | | 0 | 0 | 0 | 17.6 | 0 | 0 | |
| PFL0585w | PfpUB Plasmodium falciparum | + | 0 | 0 | 0 | 22.7 | 0 | 0 | |
| | polyubiquitin | | | | | | | | |
| PFL1515c | Chaporonin cpn60, mitochondrial | | 0 | 0 | 0 | 5.6 | 1 | 0 | |
| | precursor | | | | | | | | |
| PFC0400w | 60S Acidic ribosomal protein P2 | + | 0 | 0 | 0 | 37.5 | 0 | 1 | |
| PF14_0448 | ribosomal protein S2 | | 0 | 0 | 0 | 4 | 0 | 0 | |
| PF14_0124 | actin 11 | + | 0 | 0 | 0 | 4.3 | 0 | 1 | |
| PF07_0087 | hypothetical protein | | 0 | 0 | 0 | 9.8 | 1 | 0 | exported protein (RLE motif) |
| PF14_0627 | ribosomal protein S3 | + | 0 | 0 | 0 | 17.2 | 0 | 0 | |
| PF14_0288 | cytochromic c oxidase subunit II | | 0 | 0 | 0 | 15.7 | 0 | 0 | |
| PFE0820c | 40S ribosomal subunit protein S14 | + | 0 | 0 | 0 | 17.2 | 0 | 0 | |

*+ symbol identifies proteins found in both saponin (S) and non-saponin (NS) proteomes
**percent peptide coverage, color coding as in FIG. 2

TABLE 2

| | | SS + | | | | | | | |
|---|---|---|---|---|---|---|---|---|---|
| Gene | Name | NS | POOL 1 | POOL 2 | POOL 3 | POOL 4 | SS | TMD | Features |
| PF14_0678 | exported protein 2 (exp.2) | + | 20.2 | 10.8 | 0 | 0 | 1 | 0 | PV membrane |
| [symbol] | [symbol] | [symbol] | [symbol] | [symbol] | [symbol] | [symbol] | [symbol] | [symbol] | [symbol] |
| PF11_0175 | heat shock protein 101 | + | 19.9 | 4.6 | 0 | 0 | 1 | 0 | potential apicoplast |
| PFB0335c | cysteine protease (SERA6) | | 7.8 | 1.7 | 0 | 0 | 1 | 0 | GPI-associated (predicted) |
| PF11_0224 | exported protein 1 (exp.1) | + | 17.3 | 0 | 0 | 0 | 1 | 1 | PV membrane |
| PFB0395c | hypothetical protein | + | 15.5 | 0 | 0 | 0 | 1 | 1 | GPI-anchored (predicted; 6-cys) |
| PF11_0313 | ribosomal phosphoprotein P0 | + | 13.3 | 0 | 0 | 0 | 0 | 0 | |
| MAL5P1.71 | hypothetical protein | | 6.7 | 0 | 0 | 0 | 1 | 0 | Poss. Rhoptry surface protein |
| PFO0310w | 16 kDa sexual stage-specific | + | 7.6 | 0 | 0 | 0 | 1 | 1 | probabic gametocyte contaminant |
| | protein precursor (Pfs16 | | | | | | | | |
| [symbol] | [symbol] | [symbol] | [symbol] | [symbol] | 0 | [symbol] | [symbol] | [symbol] | [symbol] |
| PF13_0338 | hypothetical protein | + | 8.9 | 0 | 0 | 0 | 1 | 1 | GPI-anchored (predicted) |
| PF14_0344 | hypothetical protein | + | 6.0 | 0 | 0 | 1.9 | 1 | 0 | Poss. Rhoptry surface protein |
| PF13_0304 | elongation factor 1 alpha | + | 5.2 | 8.6 | 16.7 | 13.1 | 0 | 0 | |
| PF13_0305 | elongation factor 1 alpha | + | 5.2 | 8.6 | 16.7 | 13.1 | 0 | 0 | |
| PF14_0205 | ribosomal protein S25 | + | 10.4 | 0 | 18.5 | 11.1 | 1 | 0 | potential apicoplast |
| PFI0875w | Heat shock protein | + | 7.2 | 0 | 7.1 | 10.9 | 1 | 0 | |
| PFI1475w | merozoite surface protein 1 | + | 10.0 | 6.2 | 6.6 | 10.1 | 1 | 1 | GPI-anchored surface |
| PFB0300c | merozoite surface protein 2 | + | 43.8 | 34.2 | 19.5 | 0 | 1 | 1 | GPI-anchored surface |
| PF10_0366 | ADP/ATP transporter on adenylate | | 4 | 4 | 3.7 | 0 | 0 | 4 | |
| | translocase | | | | | | | | |
| PFC0725c | transporter | + | 2.9 | 6.5 | 2.9 | 2.9 | 0 | 7 | |
| PF14_0201 | hypothetical protein | + | 1 | 0 | 1 | 0 | 1 | 1 | GPI-anchored (predicted) |
| PF11_0528 | hypothetical protein | | 0.2 | 0 | 0.2 | 0 | 0 | 4 | |
| MAL13P1.130 | hypothetical protein | + | 0 | 4.3 | 5 | 0 | 1 | 6 | strong late stage exp. poss. |
| | | | | | | | | | surface protein |
| PFC0400w | 60S Acidic ribosomal protein P2 | + | 0 | 0 | 42.0 | 0 | 0 | 1 | |
| PFO1055w | ribosomal protein S19s | | 0 | 0 | 17.6 | 0 | 0 | 0 | |
| PF13_0014 | 40S ribosomal protein S7 homologue | | 0 | 0 | 11.9 | 0 | 0 | 0 | |
| PF11_0065 | ribosomal protein S4 | | 0 | 0 | 11.0 | 0 | 1 | 0 | |
| MAL6P2.254 | hypothetical protein | | 0 | 0 | 6.5 | 0 | 0 | 0 | |
| PF14_0124 | actin 11 | + | 0 | 0 | 4.3 | 0 | 0 | 1 | |
| PFD0095c | hypothetical protein | | 0 | 0 | 2.1 | 0 | 0 | 1 | exported protein (RLE motif) |
| MAL7P1.146 | hypothetical protein | + | 0 | 0 | 0.4 | 0 | 0 | 8 | |
| MAL8P1.130 | hypothetical protein | | 0.1 | 0 | 0.3 | 0 | 0 | 10 | |
| PFL0050c | hypothetical protein | | 0 | 0 | 13.2 | 6.4 | 0 | 1 | exported protein (RLE motif) |
| PFE0040c | Mature parasite infected erythrocyte | + | 0 | 0 | 4.0 | 2.1 | 1 | 0 | exported protein (RLE motif) |
| | surface antigen 1 | | | | | | | | |
| PFL0585w | PfpUB Plasmodium falciparum | + | 4.2 | 7.6 | 0 | 6.6 | 0 | 0 | |
| | polyubiquitin | | | | | | | | |
| PF14_0615 | ATP synthase (C/AC39) subunit | + | 3.7 | 3.7 | 0 | 5.8 | 0 | 0 | |
| PF13_0346 | ubiquitin/ribosomal fusion protein | + | 0 | 22.7 | 0 | 19.5 | 0 | 0 | |
| | uba52 homologue | | | | | | | | |
| PF11_0465 | dynamin-like protein | | 0 | 3.1 | 0 | 1.2 | 0 | 0 | |
| PFB0340c | cysteine protease (SERA5) | | 0 | 3.1 | 0 | 2.1 | 1 | 0 | GPI-associated (predicted) |
| MAL6P3.215 | pyridoxine biosynthetic enzyme | | 10.3 | 0 | 0 | 8.3 | 0 | 1 | |
| | pdx1 homologue | | | | | | | | |
| MAL6P1.299 | Pf12 | + | 2.9 | 0 | 0 | 3.2 | 1 | 1 | GPI-anchored (predicted 6-cys.) |
| PF14_0265 | hypothetical protein | | 0.5 | 0 | 0 | 0.8 | 0 | 1 | |
| PFD0090c | hypothetical protein | + | 0 | 0 | 7.0 | 7.0 | 0 | 1 | exported protein (RLE motif) |
| PFE1150w | multidrug resistance protein | + | 0 | 0.8 | 3.2 | 4.0 | 0 | 10 | |
| PFA0110w | ring-infected erythrocyte surface | | 0 | 0 | 1.4 | 4.2 | 0 | 1 | exported protein (RLE motif) |
| | antigen precursor | | | | | | | | |
| PFI0880c | acid phosphotase (PIGAP50) | + | 0 | 0 | 2.3 | 7.1 | 1 | 2 | inner membrane/glidosome |
| PFL2215w | actin | + | 0 | 0 | 6.4 | 15.2 | 0 | 1 | |
| PF11_0509 | ring-infected erythrocyte surface | | 0 | 0 | 1.1 | 2 | 1 | 0 | exported protein (RLE motif) |
| | antigen | | | | | | | | |
| PF13_0228 | 40S ribosomal subunit protein S6 | + | 0 | 0 | 10.8 | 29.0 | 0 | 0 | |
| MAL8P1.22 | high mobility group protein | + | 0 | 0 | 19.2 | 33.3 | 0 | 0 | |
| PF11_0507 | antigen 332 | + | 0 | 0 | 0 | 0.4 | 0 | 0 | exported protein |
| PF13_0207 | 1-dooxy-D-xylulose 5-phosphate | | 0 | 0 | 0 | 1.6 | 1 | 1 | |
| | synthase | | | | | | | | |
| PFC1020c | 40S ribosomal protein S3A | | 0 | 0 | 0 | 17.9 | 0 | 0 | |
| PF10_0084 | tubulin beta chain | | 0 | 0 | 0 | 4.9 | 0 | 2 | |
| PFI0180w | alpha tubulin | | 0 | 0 | 0 | 4.9 | 0 | 1 | |
| PFD0080c | hypothetical protein | + | 0 | 0 | 0 | 8.2 | 1 | 1 | exported protein (RLE motif) |
| PF10_0217 | pre-mRNA splicing factor | | 0 | 0 | 0 | 4.5 | 0 | 0 | |
| [symbol] | [symbol] | [symbol] | [symbol] | [symbol] | [symbol] | [symbol] | [symbol] | [symbol] | [symbol] |
| PFD1170c | hypothetical protein | + | 0 | 0 | 0 | 9.7 | 0 | 1 | exported protein (RLE motif) |
| [symbol] | [symbol] | [symbol] | [symbol] | [symbol] | [symbol] | [symbol] | [symbol] | [symbol] | [symbol] |
| PFI1780w | hypothetical protein | + | 0 | 0 | 0 | 10.7 | 1 | 0 | exported protein (RLE motif) |
| PF14_0373 | ubiquinol cytochrome | | 0 | 0 | 0 | 7.3 | 0 | 1 | |
| | c oxidoreductase | | | | | | | | |
| PF14_0597 | cytochrome c1 precursor | | 0 | 0 | 0 | 7.4 | 0 | 2 | |
| PF08_0137 | hypothetical protein | + | 0 | 0 | 0 | 9.6 | 1 | 1 | poss. exported protein (RLE motif?) |
| PFE0810c | 40S ribosomal subunit protein S14 | + | 0 | 0 | 0 | 17.2 | 0 | 0 | |
| PF11_0061 | histone H4 | | 0 | 0 | 0 | 28.2 | 0 | 0 | |
| MAL6P1.248 | histone h3 | | 0 | 0 | 0 | 14.0 | 0 | 0 | |
| PF14_0627 | ribosomal protein S3 | + | 0 | 0 | 0 | 17.6 | 0 | 0 | |
| PF10_0159 | glycophorin-binding protein 130 | | 0 | 0 | 1.7 | 10.1 | 0 | 1 | exported protein (RLE motif) |
| PF08_0054 | heat shock 70 kDa protein | + | 4.0 | 0 | 4.3 | 19.8 | 0 | 0 | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of eliciting or inducing, in a mammal, an immune response directed to a Plasmodium microorganism said method comprising administering to said mammal an effective amount of a composition which composition comprises one or more protein molecules, selected from Pf38, Pf12, Pf41, Pf92 or Pf113 or an immunogenic fragment, derivative, homologue or variant thereof, for a time and under conditions sufficient to elicit or induce an immune response to one or more of said proteins.

2. The method according to claim 1 wherein said mammal is a human.

3. The method according to claim 1 wherein said Plasmodium microorganism is selected from P. falciparum, P. malariae, P. ovale and P. vivax, P. yoelii, P. berhei, P. chabaudi, P. knowlesi, P. reichnowi, P. simium, P. fieldi, P. simiovale, P. cyanomolgi, P. hylobati, P. inui, P. gonderi, P. gallinaceum or P. elongatum.

4. The method according to claim 3 wherein said Plasmodium microorganism is selected from the Plasmodium species P. falciparum, P. malariae, P. ovale and P. vivax.

5. The method according to claim 4 wherein said Plasmodium microorganism is P. falciparum.

6. The method according to claim 1 wherein said composition comprises Pf38 or an immunogenic fragment, derivative, homologue or variant thereof.

7. The method according to claim 6 wherein said composition also comprises one or more of Pf12, Pf41, Pf92 or Pf113 or an immunogenic fragment, derivative, homologue or variant thereof.

8. The method according to claim 1 wherein said composition comprises Pf12 or an immunogenic fragment, derivative, homologue or variant thereof.

9. The method according to claim 8 wherein said composition also comprises one or more of Pf38, Pf41, Pf92 or Pf113 or an immunogenic fragment, derivative, homologue or variant thereof.

10. The method according to claim 1 wherein said composition comprises Pf38 and Pf12 or an immunogenic fragment, derivative, homologue or variant thereof.

11. A method of therapeutically or prophylactically treating a mammal for a Plasmodium microorganism infection said method comprising administering to said mammal an effective amount of a composition which composition comprises one or more protein molecules selected from Pf38, Pf12, Pf41, Pf92 or Pf113 or an immunogenic fragment, derivative, homologue or variant thereof, for a time and under conditions sufficient to elicit or induce an immune response to one or more of said proteins wherein said immune response reduces, inhibits or otherwise alleviates any one or more symptoms associated with the infection of said mammal by said Plasmodium.

12. A method for the treatment or prophylaxis of a mammalian disease condition characterized by a Plasmodium microorganism infection said method comprising administering to said mammal an effective amount of a composition which composition comprises one or more protein molecules selected from Pf38, Pf12, Pf41, Pf92 or Pf113 or an immunogenic fragment, derivative, homologue or variant thereof for a time and under conditions sufficient to elicit or induce an immune response to one or more of said proteins wherein said immune response reduces, inhibits or otherwise alleviates any one or more symptoms associated with said microorganism infection.

13. The method of claim 12 wherein said disease condition is malaria.

14. The method according to claim 11 or 12 wherein said mammal is a human.

15. The method according to claim 11 or 12 wherein said Plasmodium microorganism is selected from P. falciparum, P. malariae, P. ovale and P. vivax, P. yoelii, P. berhei, P. chabaudi, P. knowlesi, P. reichnowi, P. simium, P. fieldi, P. simiovale, P. cyanomolgi, P. hylobati, P. inui, P. gonderi, P. gallinaceum or P. elongatum

16. The method according to claim 15 wherein said Plasmodium microorganism is selected from the Plasmodium species P. falciparum, P. malariae, P. ovale and P. vivax.

17. The method according to claim 16 wherein said Plasmodium microorganism is Plasmodium falciparum.

18. The method according to claim 11 or 12 wherein said composition comprises Pf38 or an immunogenic fragment, derivative, homologue or variant thereof.

19. The method according to claim 18 wherein said composition also comprises one or more of Pf12, Pf41, Pf92 or Pf113 or an immunogenic fragment, derivative, homologue or variant thereof.

20. The method according to claim 11 or 12 wherein said composition comprises Pf12 or an immunogenic fragment, derivative, homologue or variant thereof.

21. The method according to claim 20 wherein said composition also comprises one or more of Pf12, Pf41, Pf92 or Pf113 or an immunogenic fragment, derivative, homologue or variant thereof.

22. A method according to claim 11 or 12 wherein said composition comprises Pf38 and Pf12 or an immunogenic fragment, derivative, homologue or variant thereof.

23. A composition capable of inducing an immune response to a Plasmodium microorganism said composition comprising one or more protein molecules selected from Pf38, Pf12, Pf41, Pf92 or Pf113 or an immunogenic fragment, derivative, homologue or variant thereof.

24. A composition capable of inducing an immune response to a Plasmodium microorganism comprising a vector capable of transfecting a target cell wherein the vector comprises a nucleic acid molecule capable of expressing one or more protein molecules selected from Pf38, Pf12, Pf41, Pf92 or Pf113 or an immunogenic fragment, derivative, homologue or variant thereof.

25. The composition according to claim 23 or 24 wherein said Plasmodium microorganism is selected from P. falciparum, P. malariae, P. ovale and P. vivax, P. yoelii, P. berhei, P. chabaudi, P. knowlesi, P. reichnowi, P. simium, P. fieldi, P. simiovale, P. cyanomolgi, P. hylobati, P. inui, P. gonderi, P. gallinaceum or P. elongatum

26. The composition according to claim 25 wherein said Plasmodium microorganism is P. falciparum, P. malariae, P. ovale and P. vivax.

27. The composition according to claim 26 wherein said Plasmodium microorganism is falciparum.

28. The composition according to claim 23 or 24 wherein said composition comprises Pf38 or an immunogenic fragment, derivative, homologue or variant thereof.

29. The composition according to claim 28 wherein said composition also comprises one or more of Pf12, Pf41, Pf92 or Pf113 or an immunogenic fragment, derivative, homologue or variant thereof.

30. The composition according to claim 23 or 24 wherein said composition comprises Pf12 or an immunogenic fragment, derivative, homologue or variant thereof.

31. The composition according to claim 30 wherein said composition also comprises one or more of Pf38, Pf41, Pf92 or Pf113 or an immunogenic fragment, derivative, homologue or variant thereof.

32. A composition according to claim 23 or 24 wherein said composition comprises Pf12 and Pf38 or an immunogenic fragment, derivative, homologue or variant thereof.

33. A pharmaceutical composition capable of inducing an immune response to a Plasmodium microorganism comprising the composition of claim 23 or 24 together with one or more pharmaceutically acceptable carriers and/or diluents.

34. An antibody or antibody fragment directed to one or more protein molecules selected from Pf38, Pf12, Pf41, Pf92 or Pf113 or an immunogenic fragment, derivative, homologue or variant thereof.

35. An antibody according to claim 34 wherein said antibody is a monoclonal antibody.

36. An antibody according to claim 34 wherein said antibody is a polyclonal antibody.

37. A pharmaceutical composition comprising an antibody of claim 34 together with one or more pharmaceutically acceptable carriers or diluents.

38. The composition according to claim 23 or 24 for use in therapy.

39. A method of reducing or alleviating the symptoms associated with a Plasmodium microorganism infection said method comprising administering to a mammal an effective amount of an antibody according to claim 34.

40. A method of inhibiting, halting or delaying the onset or progression of a Plasmodium microorganism infection in a mammal said method comprising administering to said mammal an effective amount of an antibody according to claim 34.

41. The method according to any one of claims 39 or 40 wherein said mammal is a human.

42. The method according to claim 39 or 40 wherein said Plasmodium microorganism infection is associated with the onset of malaria.

43. A method according to claim 42 wherein said Plasmodium microorganism is selected from the Plasmodium species P. falciparum, P. malariae, P. ovale and P. vivax.

44. The method according to claim 43 wherein said Plasmodium microorganism is P. falciparum.

45. The method according to claim 39 or 40 wherein said composition comprises an antibody directed to Pf38 or an immunogenic fragment, derivative, homologue or variant thereof.

46. The method according to claim 45 wherein said composition also comprises one or more antibodies directed to Pf12, Pf41, Pf92 or Pf113 or an immunogenic fragment, derivative, homologue or variant thereof.

47. The method according to claim 39 or 40 wherein said composition comprises an antibody directed to Pf12 or an immunogenic fragment, derivative, homologue or variant thereof.

48. The method according to claim 47 wherein said composition also comprises one or more antibodies directed to Pf38, Pf41, Pf92 or Pf113 or an immunogenic fragment, derivative, homologue or variant thereof.

49. The method according to claim 39 wherein said composition comprises antibodies directed to Pf38 and Pf12 or an immunogenic fragment, derivative, homologue or variant thereof.

50. An isolated protein as set forth in SEQ ID NOs: 2, 4, 6, 8 or 10 or having at least about 80% or greater identity to SEQ ID NOs: 2, 4, 6, 8 or 10 across the length of the sequence or a functional or immunogenic fragment or homologue thereof.

51. An isolated protein encoded by a nucleic sequence as set forth in SEQ ID NOs: 1, 3, 5, 7 or 9 or the sequence complementary to a sequence capable of hybridizing to SEQ ID NOs: 1, 3, 5, 7 or 9 under low stringency conditions and which encodes an amino acid sequence as set forth in SEQ ID NOs: 2, 4, 6, 8 or 10 or having at least about 80% or greater identity to SEQ ID NOs: 2, 4, 6, 8 or 10 across the length of the sequence.

52. An isolated nucleic acid selected from the list consisting of:
(i) An isolated nucleic acid molecule or functional fragment or homologue comprising a nucleotide sequence encoding, or complementary to a sequence encoding, an amino acid sequence substantially as set forth in SEQ ID NO: 2, 4, 6, 8 or 10 or a functional or immunogenic fragment or homologue thereof, or an amino acid sequence having at least 80%, 90%, 95%, 96%, 97%, 98%, 99% or more sequence identity to SEQ ID NO: 2, 4, 6, 8 or 10 over the length of the sequence, and/or a nucleic acid sequence capable of hybridizing to said nucleic acid molecule under low stringency conditions at 42° C.;
(ii) An isolated nucleic acid molecule or functional or immunogenic fragment or homologue thereof comprising a nucleotide sequence encoding, or complementary to said sequence, wherein said nucleotide sequence is substantially as set forth in SEQ ID NO: 1, 3, 5, 7 or 9 or a nucleotide sequence having at least about 80%, 90%, 95%, 96%, 97%, 98%, 99% or more identity over the length of the sequence of a nucleotide sequence capable of hybridizing to SEQ ID NO: 1, 3, 5, 7 or 9 or complementary form thereof under low stringency conditions at 42° C.; and
(iii) An isolated nucleic acid molecule or derivative, homologue or analogue thereof comprising a nucleotide sequence as set forth in SEQ ID NO: 1, 3, 5, 7 or 9.

53. An expression vector comprising an isolated nucleic acid according to claim 52.

54. An expression vector library wherein said expression vector library comprises the expression vector of claim 53.

55. A transformed cell expressing a nucleic acid sequence according to claim 52.
